(19) United States
(12) Patent Application Publication
(10) Pub. No.: US 2026/0363644 A1
(43) Pub. Date: Nov. 19, 2026

(54) X-RAY IMAGING APPARATUS, MEDICAL IMAGE PROCESSING APPARATUS, AND MEDICAL IMAGE PROCESSING METHOD

(71) Applicant: CANON KABUSHIKI KAISHA, Tokyo (JP)

(72) Inventors: Asateru KIMURA, Kanagawa (JP); Kosuke ARITA, Kanagawa (JP); Guang Yi ONG, Tochigi (JP); Yoshinori HIRANO, Chiba (JP)

(73) Assignee: CANON KABUSHIKI KAISHA, Tokyo (JP)

(21) Appl. No.: 19/677,415

(22) Filed: May 14, 2026

(30) Foreign Application Priority Data
May 16, 2025 (JP) 2025-082564

(51) Int. Cl. A61B 6/00; A61B 6/46

(52) U.S. Cl. CPC A61B 6/5217; A61B 6/465; A61B 6/469; A61B 6/5205

(57) ABSTRACT

An X-ray imaging apparatus and a medical image processing apparatus according to an embodiment include: an acquirer configured to acquire a medical image of a subject and at least one of site information related to an examination purpose of the subject and region information in the medical image; a setting device configured to set a first region of interest corresponding to at least one of the site information related to the examination purpose of the subject and the region information in the medical image; a specifier configured to specify a second region of interest other than the first region of interest based on the first region of interest; and a generator configured to generate a report regarding the second region of interest by using a generative model.

### CROSS REFERENCE TO RELATED APPLICATION

This application is based upon and claims the benefit of priority from the prior Japanese Patent Application No. 2025-082564, filed on May 16, 2025, the entire contents of which are incorporated herein by reference.

### FIELD

Embodiments disclosed in this specification and the drawings relate to an X-ray imaging apparatus, a medical image processing apparatus, and a medical image processing method.

### BACKGROUND

Conventionally, physicians such as radiologists perform image interpretation of medical images and create image interpretation reports to determine diagnosis and treatment effects. Findings corresponding to a request purpose (examination purpose) are described in the image interpretation reports. In addition, in a case in which a physician accidentally discovers an abnormal finding outside the examination purpose (incidental finding), the incidental finding is described in the image interpretation report. However, the incidental finding may be overlooked during image interpretation by the physician.

In addition, since creation of the image interpretation report imposes a burden on a physician, for example, there is known a technique for automatically generating an image interpretation report regarding a region of a medical image to which the physician pays attention. However, an incidental finding may be included also in a region other than the region to which the physician pays attention, and in a conventional technique, there is a possibility that the incidental finding is not included in an image interpretation report that is automatically generated.

### BRIEF DESCRIPTION OF THE DRAWINGS

FIG. 1 is a block diagram illustrating a configuration example of a medical image processing system according to a first embodiment;

FIG. 2 is a block diagram illustrating a configuration example of a medical image processing apparatus according to the first embodiment;

FIG. 3 is a flowchart illustrating an example of report generation processing in the medical image processing system according to the first embodiment;

FIG. 4 is a diagram schematically illustrating an example of a process up to a step of specifying a region of non-interest in the medical image processing system according to the first embodiment;

FIG. 5 is a diagram schematically illustrating an example of a step of generating a prompt related to the region of non-interest in the medical image processing system according to the first embodiment;

FIG. 6 is a diagram schematically illustrating an example of a step of generating a prompt related to a region of interest in the medical image processing system according to the first embodiment;

FIG. 7 is a diagram schematically illustrating an example of a step of generating a report regarding the region of non-interest in the medical image processing system according to the first embodiment;

FIG. 8 is a diagram illustrating an example of a report in the medical image processing system according to a reference example;

FIG. 9 is a diagram illustrating another example of a report in the medical image processing system according to the reference example;

FIG. 10 is a diagram illustrating an example of a report output screen in the medical image processing system according to the first embodiment;

FIG. 11 is a diagram illustrating details of a report regarding the region of non-interest in FIG. 10;

FIG. 12 is a diagram illustrating details of a report regarding the region of interest in FIG. 10;

FIG. 13 is a diagram illustrating details of a new report in FIG. 10;

FIG. 14 is a block diagram illustrating a configuration example of a medical image processing system according to a modification of the first embodiment;

FIG. 15 is a diagram schematically illustrating an example of a step of generating a prompt related to a region of non-interest in the medical image processing system according to the modification of the first embodiment;

FIG. 16 is a block diagram illustrating a configuration example of a medical image processing apparatus according to a second embodiment;

FIG. 17 is a flowchart illustrating an example of report generation processing in a medical image processing system according to the second embodiment;

FIG. 18 is a diagram schematically illustrating an example of a process up to a step of specifying a region of non-interest in the medical image processing system according to the second embodiment;

FIG. 19 is a diagram schematically illustrating another example of a step of specifying a region of non-interest in the medical image processing system according to the second embodiment;

FIG. 20 is a diagram schematically illustrating an example of a step of generating a prompt in the medical image processing system according to the second embodiment and a step of generating a report regarding a region of non-interest;

FIG. 21 is a diagram illustrating an example of a report output screen in the medical image processing system according to the second embodiment;

FIG. 22 is a flowchart illustrating an example of report generation processing in a medical image processing system according to a modification of the second embodiment;

FIG. 23 is a diagram schematically illustrating an example of a process up to a step of specifying a region of non-interest in the medical image processing system according to the modification of the second embodiment;

FIG. 24 is a diagram schematically illustrating an example of a process up to a step of specifying a region of non-interest in the medical image processing system according to the modification of the second embodiment subsequent to FIG. 23;

FIG. 25 is a diagram illustrating an example of a report output screen in the medical image processing system according to the modification of the second embodiment;

FIG. 26 is a block diagram illustrating a configuration example of a medical image processing apparatus according to a third embodiment;

FIG. 27 is a flowchart illustrating an example of report verification processing in a medical image processing system according to the third embodiment;

FIG. 28 is a diagram illustrating an example of a report verification screen in the medical image processing system according to the third embodiment;

FIG. 29 is a diagram illustrating details of a verification report regarding a region of non-interest in FIG. 28;

FIG. 30 is a diagram illustrating details of a verification report regarding a region of interest in FIG. 28;

FIG. 31 is a diagram illustrating details of a new verification report in FIG. 28;

FIG. 32 is a block diagram illustrating a configuration example of a medical image processing apparatus according to a fourth embodiment;

FIG. 33 is a block diagram illustrating a configuration example of a medical image processing system according to a fifth embodiment; and

FIG. 34 is a flowchart illustrating an example of report generation processing in the medical image processing system according to the fifth embodiment.

### DETAILED DESCRIPTION

The following describes embodiments of an X-ray imaging apparatus, a medical image processing apparatus, and a medical image processing method with reference to the drawings. In the following description, constituent elements having substantially the same functions and configurations are denoted by the same reference numerals, and redundant description will be provided only when necessary.

### First embodiment

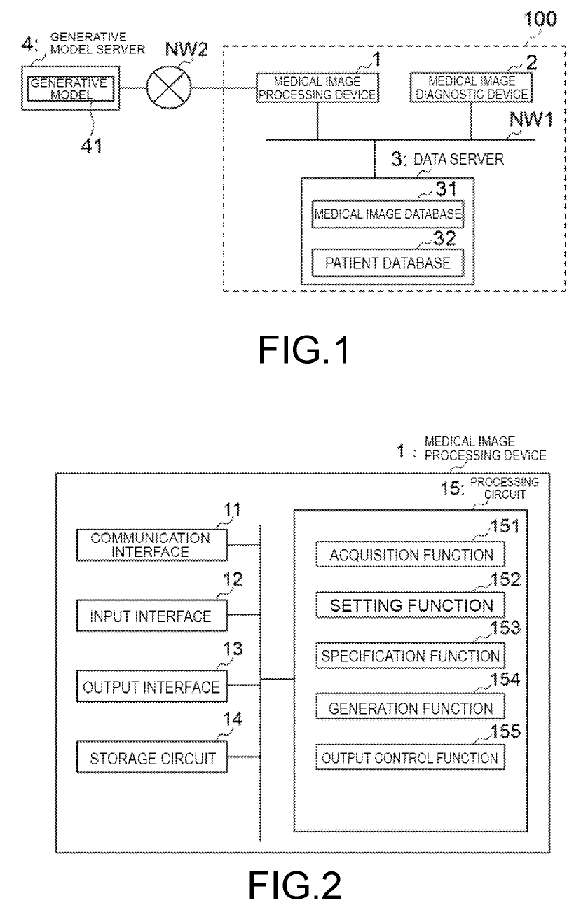

FIG. 1 is a block diagram illustrating a configuration example of a medical image processing system 100 according to a first embodiment. As illustrated in FIG. 1, the medical image processing system 100 according to the present embodiment includes a medical image processing apparatus 1, a medical image diagnostic apparatus 2, and a data server 3. The medical image processing apparatus 1, the medical image diagnostic apparatus 2, and the data server 3 are connected to each other in a communicable manner via a communication network NW1. The medical image processing apparatus 1 is connected to a generative model server 4 in a communicable manner via a communication network NW2. In the example of FIG. 1, the generative model server 4 is not included in the medical image processing system 100.

The medical image processing apparatus 1 is a apparatus that processes a medical image acquired by imaging a subject such as a patient. The medical image processing apparatus 1 is, for example, a workstation, an image interpretation terminal, and the like, and may be operated by a user such as a physician or a patient. For example, the medical image processing apparatus 1 generates, based on a medical image of a subject such as a patient, an image interpretation report regarding the medical image.

The medical image diagnostic apparatus 2 is a apparatus that generates a medical image by imaging a subject. The medical image diagnostic apparatus 2 is, for example, an X-ray imaging apparatus (X-ray diagnostic apparatus) such as a general X-ray diagnostic apparatus and an X-ray Computed Tomography (CT) apparatus, a Magnetic Resonance Imaging (MRI) apparatus, an ultrasonic diagnostic apparatus, a Single Photon Emission Computed Tomography (SPECT) apparatus, a Positron Emission Tomography (PET) apparatus, and the like. The medical image diagnostic apparatus 2 that is an X-ray imaging apparatus, an MRI apparatus, or an ultrasonic diagnostic apparatus images a subject, and respectively generates an X-ray image, an MRI image, or an ultrasonic image as a medical image. The present embodiment describes a case in which the medical image diagnostic apparatus 2 is an X-ray imaging apparatus as an example.

The data server 3 is a server that stores medical data and the like generated by the medical image processing apparatus 1 or the medical image diagnostic apparatus 2. The data server 3 is, for example, an image server such as a Picture Archiving and Communication System (PACS). The data server 3 may be configured in the medical image processing apparatus 1, or may be provided on a cloud.

As illustrated in FIG. 1, in the present embodiment, the data server 3 includes a medical image database 31 and a patient database 32.

The medical image database 31 stores, for example, a medical image generated by the medical image diagnostic apparatus 2 and the like. The medical image database 31 may store a medical image acquired from a database in a hospital such as a PACS or a database for machine learning, or may store a medical image acquired from the Internet or the like.

The patient database 32 stores various kinds of patient information related to the patient. The patient information includes, for example, information such as an age, a sex, a height, and a weight of the patient, and information related to a disease name with which the patient is affected and a treatment state. The patient database 32 may store information of an electronic medical chart.

The communication network NW1 is used for communication among the medical image processing apparatus 1, the medical image diagnostic apparatus 2, and the data server 3. In the present embodiment, the communication network NW1 is an in-hospital network based on a dedicated line in a hospital. The communication network NW1 may be an external network such as the Internet.

The generative model server 4 is, for example, a cloud server on the communication network NW2. The generative model server 4 includes a generative model 41, which is an example of a foundation model. The generative model 41 has a function of outputting information about an image interpretation report based on an input prompt. The generative model 41 is, for example, a Multimodal Model (MMM) that can receive inputs of character strings and images as prompts. The generative model server 4 is not limited to the cloud server and may be a server device installed in a hospital. In this case, the generative model server 4 may be connected to the communication network NW1. In addition, in this case, the generative model server 4 may be included in the medical image processing system 100. The generative model server 4 may also include a plurality of generative models.

The communication network NW2 is used for communication between the medical image processing apparatus 1 and the generative model server 4. In the present embodiment, the communication network NW2 is an external network such as the Internet.

Next, the following describes the medical image processing apparatus 1 according to the present embodiment in detail with reference to FIG. 2. FIG. 2 is a block diagram illustrating a configuration example of the medical image processing apparatus 1 according to the first embodiment. As illustrated in FIG. 2, the medical image processing apparatus 1 according to the present embodiment includes a communication interface 11, an input interface 12, an output interface 13, a storage circuit 14, and a processing circuit 15.

The communication interface 11 implements various communication protocols corresponding to forms of the communication network NW1 and the communication network NW2. The communication interface 11 achieves communication with other devices via the communication networks NW1 and NW2 in accordance with the communication protocols. In the present embodiment, the medical image processing apparatus 1 communicates with the medical image diagnostic apparatus 2 and the data server 3 via the communication interface 11 and the communication network NW1. In addition, the medical image processing apparatus 1 communicates with the generative model server 4 via the communication interface 11 and the communication network NW2.

The input interface 12 receives various input operations from an operator, and converts the received input operations into electric signals to be output to the processing circuit 15. The input interface 12 includes, for example, a mouse, a keyboard, a trackball, a switch, a button, a joystick, a touch panel, and the like. The input interface 12 may be, for example, a user interface such as a microphone that receives a voice input. In a case in which the input interface 12 is a touch panel, the input interface 12 may also have a display function of a display included in the output interface 13 described later.

The input interface 12 is not limited to an interface including a physical operation component such as a mouse or a keyboard. For example, examples of the input interface 12 include an electric signal processing circuit that receives an electrical signal corresponding to an input operation from an external input device that is provided separately from the medical image processing apparatus 1 and outputs the electrical signal to the processing circuit 15.

The output interface 13 includes, for example, a display and a speaker. The output interface 13 corresponds to an output device in the present embodiment. The display included in the output interface 13 corresponds to a display device in the present embodiment. The display displays various pieces of information. For example, the display displays a screen generated by an output control function 155 of the processing circuit 15, a Graphical User Interface (GUI) for receiving various input operations from an operator, or the like. The display is, for example, a Liquid Crystal Display (LCD), a Cathode Ray Tube (CRT) display, an organic Electro Luminescence (EL) display, or the like. The speaker outputs information input via the processing circuit 15 as a voice.

The storage circuit 14 is a storage device that stores various pieces of information, and is implemented by, for example, a volatile storage device such as a Random Access Memory (RAM), a semiconductor memory element such as a flash memory, and a non-transitory storage device such as a hard disk and an optical disc. For example, the storage circuit 14 receives and stores various pieces of data and the like collected by the processing circuit 15. In addition, the storage circuit 14 stores programs corresponding to various functions executed by the processing circuit 15. The storage circuit 14 may be implemented by a server group (cloud) connected to the medical image processing apparatus 1 via a network. Various pieces of data treated in the present specification are typically digital data.

The processing circuit 15 is a control circuit that performs overall control of the medical image processing apparatus 1, and is also an arithmetic circuit that performs various arithmetic operations. In the present embodiment, the processing circuit 15 includes an acquisition function 151, a setting function 152, a specification function 153, a generation function 154, and the output control function 155.

The acquisition function 151 acquires various pieces of information from a user, the data server 3, the generative model server 4, and the like. For example, the acquisition function 151 receives an input from a user via the input interface 12. The acquisition function 151 also acquires a medical image of a subject from the medical image database 31 or the like of the data server 3. The acquisition function 151 corresponds to an acquirer in the present embodiment.

In addition, the acquisition function 151 acquires at least one of site information related to an examination purpose of a subject and region information in a medical image. In the present embodiment, the acquisition function 151 acquires the site information related to the examination purpose of the subject in a text format (character strings). The site information explicitly or implicitly includes, for example, information about a site corresponding to a request purpose of an image interpretation report. Specifically, the site information includes, as explicit information about a site, information about an organ such as a heart or a liver and information about a region such as an upper part of a right lung field. In addition, the site information includes, as explicit information about a site, information such as "examination of nausea". In the following description, a "site" includes an organ, a tissue, a region, and the like, including a portion thereof.

In the present embodiment, the acquisition function 151 acquires imaging target information related to an imaging target of a medical image. The imaging target information includes, for example, information of a site imaged in the medical image. In the present embodiment, the acquisition function 151 acquires imaging target information in a text format. The imaging target information is set based on an imaging order when the medical image is taken by using the medical image diagnostic apparatus 2. For example, the acquisition function 151 acquires the imaging target information from the imaging order based on a rule. Specifically, in a case in which chest imaging is performed by using an X-ray imaging apparatus, lungs, a heart, a liver, a pancreas, and the like are typically included in the imaging target information. Thus, in a case in which the imaging order is chest imaging, the acquisition function 151 acquires lungs, a heart, a liver, a pancreas, and the like as the imaging target information. The imaging target information may be detected from the medical image by using a learning model and the like for detecting a site included in the medical image. The imaging target information may be embedded in the medical image in advance, or may be stored in the medical image database 31 in association with the medical image.

The setting function 152 sets a region of interest corresponding to at least one of the site information related to the examination purpose of the subject and the region information in the medical image. In the present embodiment, the setting function 152 sets a region of interest in a text format based on the site information related to the examination purpose of the subject. The setting function 152 corresponds to a setting device in the present embodiment. The region of interest corresponds to a first region of interest in the present embodiment.

The specification function 153 specifies a region of non-interest other than the region of interest based on the region of interest set by the setting function 152. The region of non-interest is a region to which no attention is paid in either the site information or the region information. In the present embodiment, the specification function 153 specifies the region of non-interest in a text format. The specification function 153 corresponds to a specifier in the present embodiment. The region of non-interest corresponds to a second region of interest in the present embodiment.

The generation function 154 generates a report regarding the region of non-interest using the generative model 41. More specifically, the generation function 154 generates a prompt for generating a report regarding the region of non-interest and inputs the prompt to the generative model 41, and generates the report regarding the region of non-interest based on an output result from the generative model 41. The generation function 154 corresponds to a generator in the present embodiment.

In the present embodiment, the generation function 154 generates a report regarding the region of interest in addition to the report regarding the region of non-interest using the generative model 41. Furthermore, in the present embodiment, the generation function 154 generates a new report based on the report regarding the region of interest and the report regarding the region of non-interest.

The output control function 155 controls the output interface 13 to output various pieces of information. In the present embodiment, the output control function 155 causes the report regarding the region of non-interest to be output to the display of the output interface 13. More specifically, the output control function 155 generates a screen including the report regarding the region of non-interest, and causes the screen to be output to the display. The output control function 155 corresponds to an output control device in the present embodiment.

Herein, processing functions executed by the acquisition function 151, the setting function 152, the specification function 153, the generation function 154, and the output control function 155, which are constituent elements of the processing circuit 15 illustrated in FIG. 2, are recorded in the storage circuit 14 in a form of a computer-executable program, for example. The processing circuit 15 is, for example, a processor. The processor constituting the processing circuit 15 reads out a program from the storage circuit 14, and executes the read-out program to implement a function corresponding to the program. In other words, the processing circuit 15 after reading out the program has a function illustrated in the processing circuit 15 in FIG. 2.

FIG. 2 illustrates a case in which the acquisition function 151, the setting function 152, the specification function 153, the generation function 154, and the output control function 155 are implemented by the single processing circuit 15, but the embodiment is not limited thereto. For example, the processing circuit 15 may be configured by combining a plurality of independent processors, and may implement these functions when each of the processors executes each of the programs. The processing functions of the processing circuit 15 may be implemented by being appropriately distributed or integrated into a single processing circuit or a plurality of processing circuits.

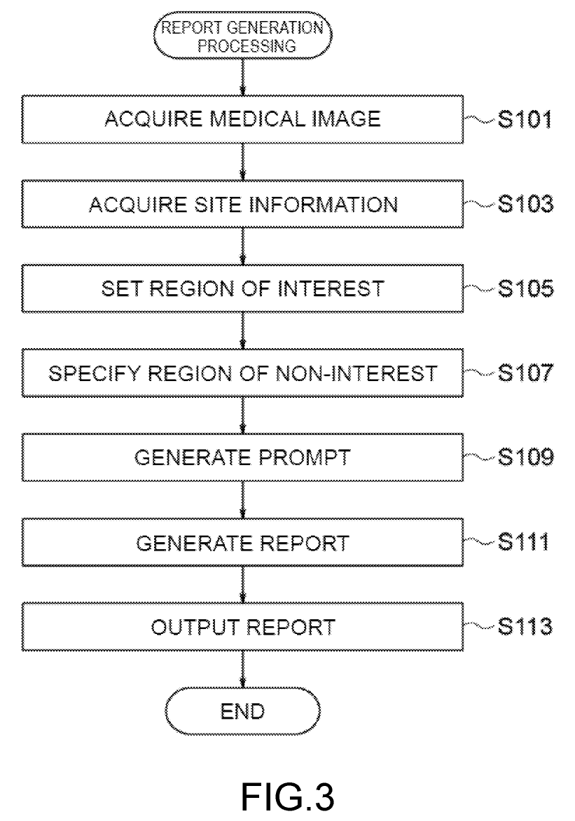

Next, with reference to FIG. 3, the following describes an example of report generation processing in the medical image processing system 100 configured as described above. FIG. 3 is a flowchart illustrating an example of the report generation processing in the medical image processing system 100 according to the first embodiment. The report generation processing is, for example, performed when a user such as a physician generates an image interpretation report by using the medical image processing system 100.

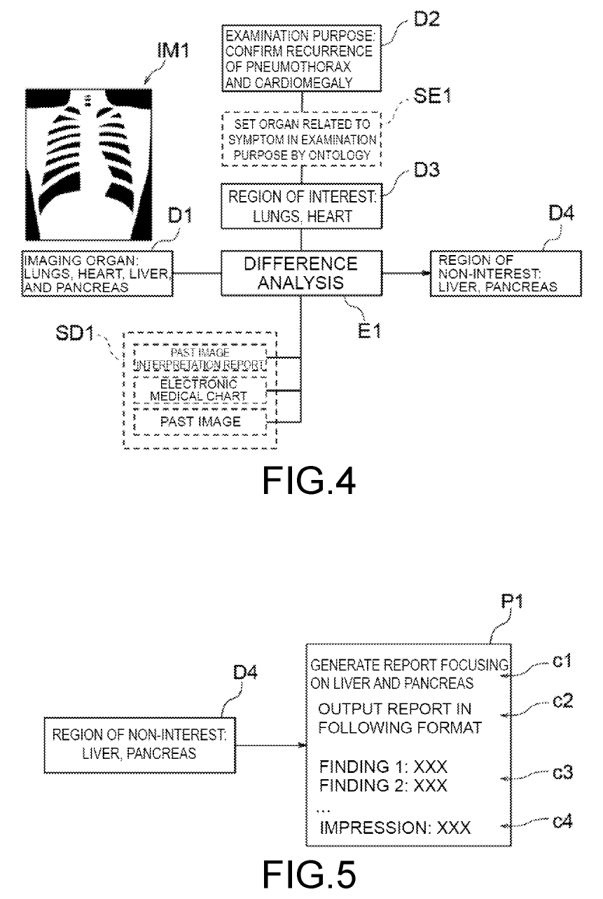

First, as illustrated in FIG. 3, the acquisition function 151 of the medical image processing apparatus 1 acquires a medical image (step S101). FIG. 4 is a diagram schematically illustrating an example of a process up to a step of specifying a region of non-interest in the medical image processing system 100 according to the first embodiment. As illustrated in FIG. 4, the acquisition function 151 of the processing circuit 15 acquires a medical image IM1 of a subject. The acquisition function 151 also acquires imaging target information D1 of the medical image IM1 in a text format. In the example of FIG. 4, the imaging target information D1 of the medical image IM1 includes, as imaging organs, information about lungs, a heart, a liver, and a pancreas in a text format. For example, the imaging target information D1 includes character strings such as "lungs, heart, liver, pancreas".

Next, as illustrated in FIG. 3, the acquisition function 151 of the medical image processing apparatus 1 acquires site information (step S103). As illustrated in FIG. 4, the acquisition function 151 of the processing circuit 15 acquires site information D2 related to an examination purpose of the subject in a text format. In the example of FIG. 4, the site information D2 includes, as information about an examination purpose, character strings such as "confirm recurrence of pneumothorax and cardiomegaly".

Next, as illustrated in FIG. 3, the setting function 152 of the medical image processing apparatus 1 sets a region of interest (step S105). As illustrated in FIG. 4, the setting function 152 of the processing circuit 15 sets a region of interest D3 in a text format based on the site information D2. The setting function 152 sets the region of interest D3 by extracting a name of a site from the site information D2 based on a named entity or a keyword, for example. In the example of FIG. 4, the region of interest D3 includes information about lungs and a heart in a text format. For example, the region of interest D3 includes character strings of "lungs, heart".

Setting of the region of interest D3 by the setting function 152 of the processing circuit 15 may include setting processing SE1 using ontology. The ontology is, for example, generated in advance, and stored in the storage circuit 14. The ontology has a graph structure in which, for example, medical concepts related to a disease, a symptom, or a treatment method are nodes, and relations between the medical concepts are edges. In the setting processing SE1, based on information about the symptom of the subject included in the site information D2, the setting function 152 sets the region of interest D3 related to the symptom using the ontology. Accordingly, in a case in which the site information D2 does not include explicit information about the site, the setting function 152 can set the region of interest D3. However, the setting processing SE1 is not necessarily provided.

Next, as illustrated in FIG. 3, the specification function 153 of the medical image processing apparatus 1 specifies a region of non-interest (step S107). As illustrated in FIG. 4, the specification function 153 of the processing circuit 15 specifies a region of non-interest D4 in a text format based on the region of interest D3 set by the setting function 152. More specifically, the specification function 153 specifies the region of non-interest D4 based on an imaging target, which is not included in the region of interest D3, in the imaging target information D1. In other words, the specification function 153 specifies the region of non-interest D4 as a character string of a non-corresponding site between the imaging target information D1 and the region of interest D3 by difference analysis E1 between the imaging target information D1 and the region of interest D3. Thus, in the present embodiment, it can be said that the region of non-interest D4 is a character string representing a finding or a site that is unlikely to be assumed from the examination purpose or the like included in the site information D2. In other words, it can also be said that the region of non-interest D4 is a region to which attention is newly paid based on the region of interest D3. In the example of FIG. 4, the region of non-interest D4 includes, in a text format, information about a liver and a pancreas as imaging targets not included in the region of interest D3 among sites included in the imaging target information D1. For example, the region of non-interest D4 includes character strings of "liver, pancreas".

The specification function 153 of the processing circuit 15 may specify the region of non-interest D4 based on subject information SD1 related to the subject in addition to the imaging target information D1 and the region of interest D3. The subject information SD1 is a past image interpretation report regarding the subject, an electronic medical chart of the subject, a past medical image related to the subject, and the like stored in the patient database 32, for example. The specification function 153 adds a new site to the region of non-interest D4 or deletes a site from the region of non-interest D4 based on the subject information SD1. Specifically, the specification function 153 extracts a name of a site from the subject information SD1 based on a named entity and a keyword, for example. Subsequently, the specification function 153 adds a character string related to a site not included in a past image interpretation report to the region of non-interest D4. Alternatively, the specification function 153 adds a character string related to a site included in a past image interpretation report to the region of non-interest D4. By using the subject information SD1, information about the subject can be utilized. The subject information SD1 is not necessarily used.

Next, as illustrated in FIG. 3, the generation function 154 of the medical image processing apparatus 1 generates a prompt (step S109). FIG. 5 is a diagram schematically illustrating an example of a step of generating a prompt related to the region of non-interest in the medical image processing system 100 according to the first embodiment. As illustrated in FIG. 5, the generation function 154 of the processing circuit 15 generates a prompt P1 for generating a report regarding the region of non-interest D4 based on the region of non-interest D4.

Herein, the prompt P1 includes instruction contents c1 to c4, for example. The instruction content c1 is an instruction to generate a report including sites (a liver and a pancreas) related to the region of non-interest D4. The instruction content c2 is an instruction related to an output format. The instruction content c3 is an instruction to cause a finding to be described in an output. The findings are, for example, described comprehensively for each site and correspond to contents related to respective diagnoses and conclusions for each site. The instruction content c4 is an instruction to cause an impression to be described in an output. The impression is, for example, described as an overall conclusion and a determination category for an examination purpose included in the site information D2 by comprehensively considering findings for each site.

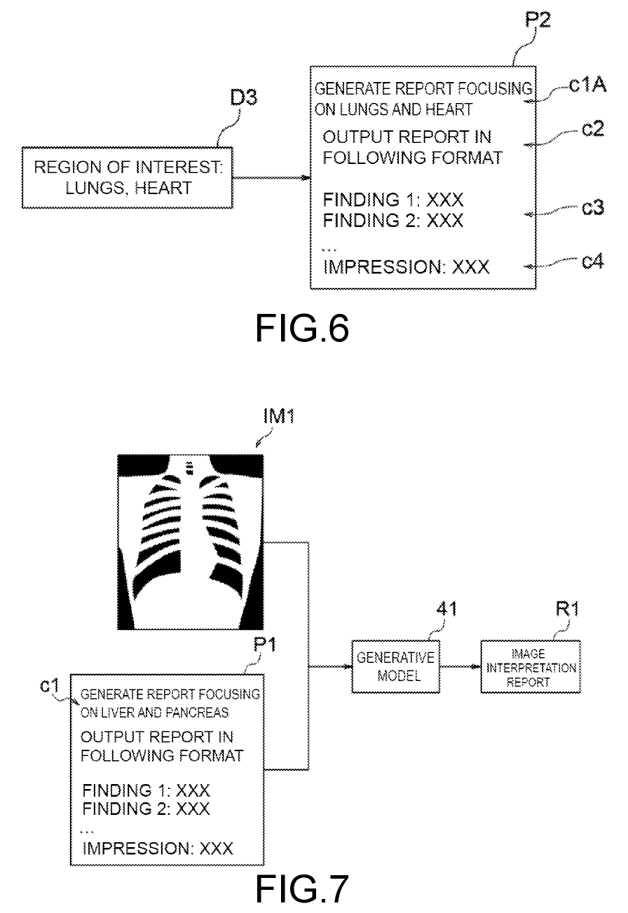

FIG. 6 is a diagram schematically illustrating an example of a step of generating a prompt related to the region of interest D3 in the medical image processing system according to the first embodiment. In the present embodiment, at step S109, the generation function 154 of the processing circuit 15 further generates a prompt P2 for generating a report regarding the region of interest D3 based on the region of interest D3. The prompt P2 includes, for example, instruction contents c1A, and c2 to c4. The instruction content c1A is an instruction to generate a report including sites (lungs and a heart) related to the region of interest D3. The instruction contents c2 to c4 are the same as those of the prompt P1 described above.

In this manner, the generation function 154 of the processing circuit 15 generates the prompt P1 related to the region of non-interest D4 and the prompt P2 related to the region of interest D3 separately. Accordingly, a report regarding the region of non-interest D4 and a report regarding the region of interest D3 can be separately generated at a subsequent step. The generation function 154 may generate a prompt so that one prompt includes both an instruction to generate a report regarding the region of non-interest D4 and an instruction to generate a report regarding the region of interest D3. In this case, the generation function 154 may cause the one prompt to include an instruction to generate a report regarding the region of non-interest D4 and a report regarding the region of interest D3 in a distinguishable manner.

Next, as illustrated in FIG. 3, the generation function 154 of the medical image processing apparatus 1 generates a report (step S111). FIG. 7 is a diagram schematically illustrating an example of a step of generating a report regarding the region of non-interest D4 in the medical image processing system 100 according to the first embodiment.

As illustrated in FIG. 7, first, the generation function 154 of the processing circuit 15 transmits the prompt P1 to the generative model 41 of the generative model server 4 via the communication interface 11. In the present embodiment, the generation function 154 transmits the medical image IM1 acquired by the acquisition function 151 of the processing circuit 15 to the generative model 41 simultaneously with the prompt P1. Accordingly, an instruction to generate a report R1, which is an image interpretation report regarding the region of non-interest D4 in the medical image IM1, is input to the generative model 41 based on the medical image IM1 and the prompt P1. Subsequently, the generation function 154 receives an output from the generative model 41 of the generative model server 4 via the communication interface 11. Thereafter, the generation function 154 generates the report R1 based on the received output from the generative model 41. The generation function 154 may change the generative model 41 to which the prompt P1 is input depending on a site included in the medical image IM1 and the site information D2 related to the examination purpose of the subject.

Similarly, the generation function 154 of the processing circuit 15 inputs the prompt P2 illustrated in FIG. 6 to the generative model 41 together with the medical image IM1, and generates a report regarding the region of interest D3 based on an output from the generative model 41. The generation function 154 may input the prompt P1 and the prompt P2 to generative models different from each other.

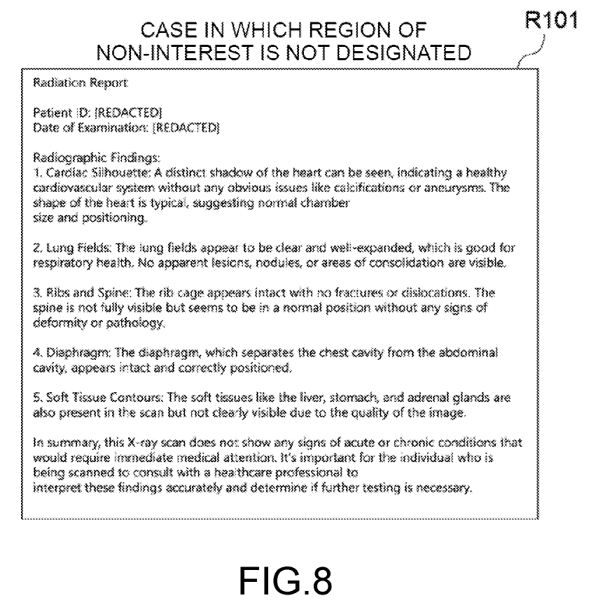

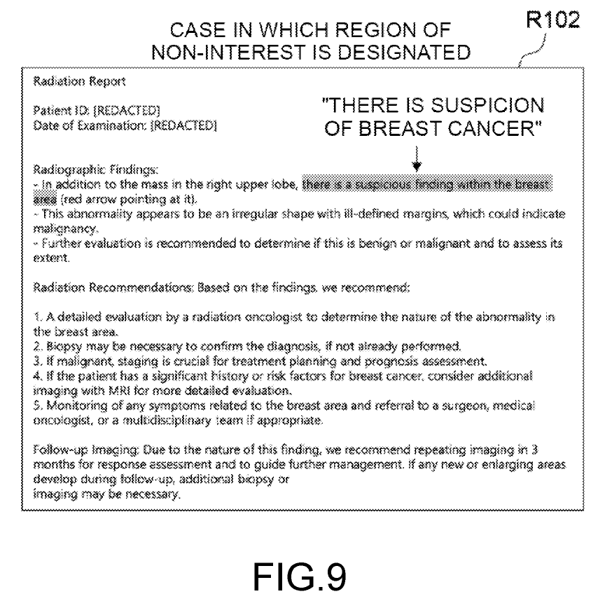

Herein, the following describes an effect of the prompt P1 including the instruction content c1 with reference to FIGS. 8 and 9. FIG. 8 is a diagram illustrating an example of a report in the medical image processing system according to a reference example. FIG. 9 is a diagram illustrating another example of a report in the medical image processing system according to the reference example.

FIG. 8 illustrates a report R101 output from the generative model when a certain medical image and a prompt that does not designate a region of non-interest are input to the generative model. The medical image is, for example, a slice image including a chest of the subject. The prompt that does not designate the region of non-interest is, for example, "create radiation report about the following image".

On the other hand, FIG. 9 illustrates a report R102 output from the generative model when the same medical image as that in a case of FIG. 8 and a prompt designating "chest" as the region of non-interest are input to the generative model. The prompt that designates the region of non-interest is, for example, "create radiation report to pay attention to chest about following image". Among these, description of "to pay attention to chest" is designation to pay attention to a "chest", which is the region of non-interest.

As illustrated in FIG. 9, the report R102 in a case in which the region of non-interest is designated includes, as an example of an incidental finding, description indicating "there is a suspicion of chest cancer". On the other hand, as illustrated in FIG. 8, the report R101 in a case in which the region of non-interest is not designated does not include such description. Based on these results, it is suggested that, by designating a region to which the generative model should pay attention, such as a region of non-interest, by a prompt, a likelihood that a finding included in that region is included in the report increases. That is, by designating the region to which the generative model should pay attention by a prompt in a form such as the instruction content c1, accuracy of the image interpretation report can be improved.

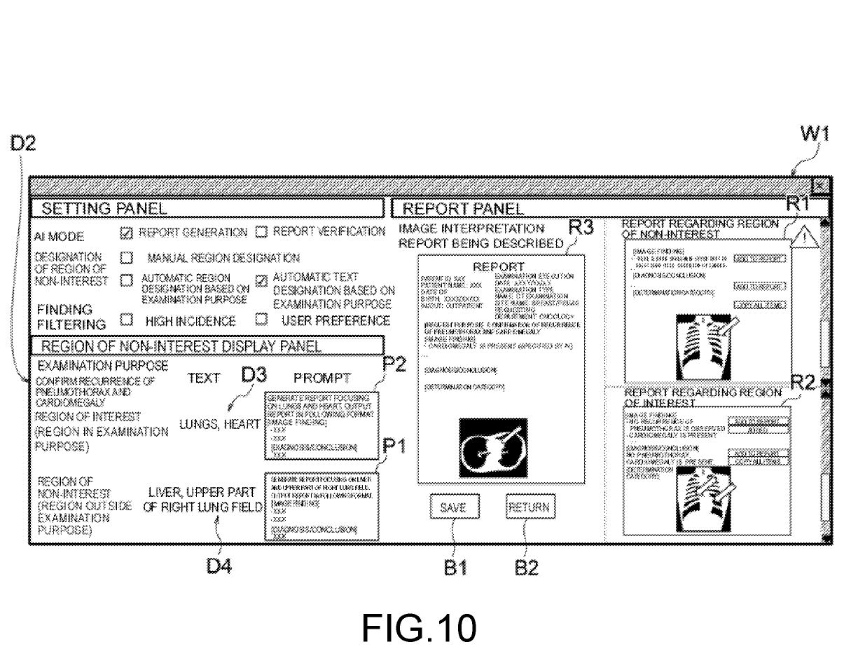

Next, as illustrated in FIG. 3, the output control function 155 of the medical image processing apparatus 1 outputs a report (step S113). FIG. 10 is a diagram illustrating an example of a report output screen in the medical image processing system 100 according to the first embodiment. The output control function 155 of the processing circuit 15 causes a screen W1 illustrated in FIG. 10 to be output to the display, for example.

As illustrated in FIG. 10, on the screen W1, a "setting panel", a "region of non-interest display panel", and a "report panel" are displayed, for example.

The "setting panel" includes "AI mode", "designation of region of non-interest", and "finding filtering". The medical image processing apparatus 1 receives an instruction regarding an operation mode of the medical image processing apparatus 1 via selection of each checkbox in the "setting panel" by a user. Accordingly, the user can easily select and grasp the operation mode of the medical image processing apparatus 1.

More specifically, in the example of FIG. 10, "report generation" is selected as "AI mode", and an operation mode for performing the report generation processing illustrated in FIG. 3 is selected. In addition, "automatic text designation based on examination purpose" is selected as "designation of region of non-interest", and an operation mode is selected in which the specification function 153 of the processing circuit 15 specifies the region of non-interest D4 in a text format from the site information D2 including the examination purpose. In the example of FIG. 10, no item in "finding filtering" is selected. Details of a case in which "report verification" is selected as "AI mode", a case in which "automatic region designation based on examination purpose" or "automatic text designation based on examination purpose" is selected as "designation of region of non-interest", and a case in which at least one item of "finding filtering" is selected will be described later.

On the "region of non-interest display panel", the site information D2, the region of interest D3, the region of non-interest D4, the prompt P1, and the prompt P2 are displayed. Accordingly, a user can easily grasp contents of the site information D2, the region of interest D3, the region of non-interest D4, the prompt P1, and the prompt P2. The medical image processing apparatus 1 may receive an input for them from a user. That is, a user may be allowed to directly edit sites included in the site information D2, the region of interest D3, and the region of non-interest D4, or contents of the prompt P1 and the prompt P2 on the screen W1. Accordingly, a user can easily edit contents of the site information D2, the region of interest D3, the region of non-interest D4, the prompt P1, and the prompt P2.

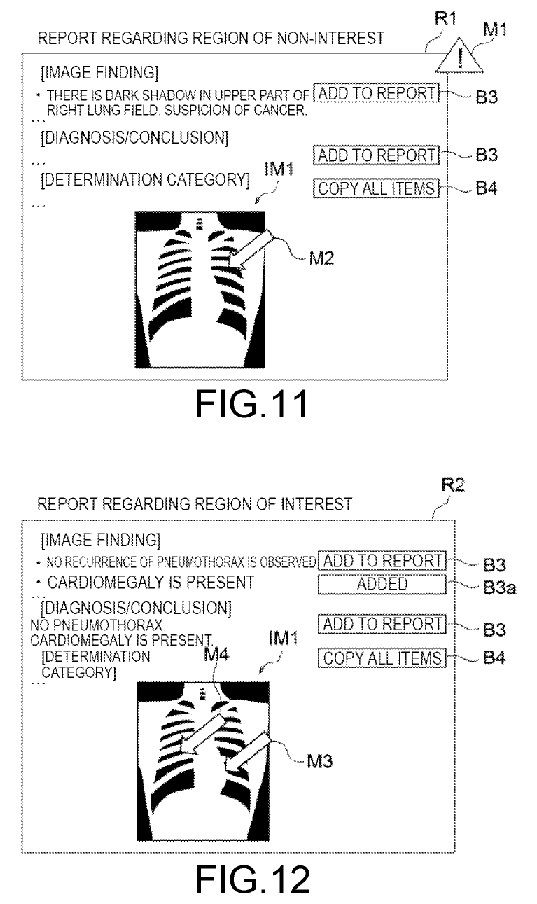

Reports R1 to R3 are displayed on the "report panel". Each of the reports R1 to R3 is an example of an image interpretation report. The report R1 is a report regarding the region of non-interest D4 described above. The report R2 is a report regarding the region of interest D3. In this manner, the output control function 155 of the processing circuit 15 causes the report R1 regarding the region of non-interest and the report R2 regarding the region of interest to be displayed on the display in an identifiable manner. Accordingly, for example, a user can easily grasp differences between the report R1 and the report R2. The report R3 is a new report that is generated based on the report R2 regarding the region of interest and the report R1 regarding the region of non-interest. The following describes details of the reports R1 to R3 with reference to FIGS. 11-13.

FIG. 11 is a diagram illustrating details of the report R1 regarding the region of non-interest D4 in FIG. 10. In the report R1, for example, "image finding", "diagnosis / conclusion", and "determination category" are described. In the example of FIG. 11, "image finding" includes a description of "there is dark shadow in upper part of right lung field. Suspicion of cancer". Thus, a user can easily grasp "image finding", "diagnosis / conclusion", and "determination category".

In the report R1 illustrated in FIG. 11, the medical image IM1, a mark M1, a mark M2, a button B3, and a button B4 may be displayed. The mark M1 is displayed, for example, as an alert mark and the like in a case in which one or more findings are included in the report R1 regarding the region of non-interest. In addition, the mark M2 is displayed together with the medical image IM1, and indicates the region of non-interest D4 on the medical image IM1 by an arrow, for example. In this manner, the output control function 155 of the processing circuit 15 causes the marks M1, M2, or the like to be displayed to emphasize a portion related to the region of non-interest D4 in the report R1. By causing the marks M1, M2, or the like to be displayed, a user can easily grasp findings included in the region of non-interest D4 and a possibility of a new finding. The mark M1 may be displayed in the report R3.

Furthermore, when a user presses the button B3, a description or a text corresponding to each button B3 is added (copied) to the report R3. Due to this, a user can easily reflect each description in the report R3. When a user presses the button B4, all descriptions generated by the generative model 41 in the report R1 (an image finding, diagnosis / conclusion, a determination category, and the like) are added to the report R3. Due to this, a user can easily reflect all descriptions in the report R1 in the report R3.

FIG. 12 is a diagram illustrating details of the report R2 regarding the region of interest in FIG. 10. In the report R2, for example, "image finding", "diagnosis / conclusion", and "determination category" are described similarly to the report R1. In the example of FIG. 12, "image finding" includes descriptions of "No recurrence of pneumothorax is observed" and "Cardiomegaly is present". In addition, "diagnosis / conclusion" includes a description of "No pneumothorax. Cardiomegaly is present".

In the report R2 illustrated in FIG. 12, the medical image IM1, a mark M3, a mark M4, the button B3, a button B3a, and the button B4 may be displayed. The button B3 and the button B4 are the same as those in the report R1. The mark M3 and the mark M4 are displayed together with the medical image IM1, and each indicate the region of interest D3 on the medical image IM1 by an arrow, for example. By causing the marks M3, M4, or the like to be displayed, a user can easily grasp findings included in the region of interest D3. The button B3a is displayed, for example, when a user presses the button B3, and represents that the description or text has been already added to the report R3.

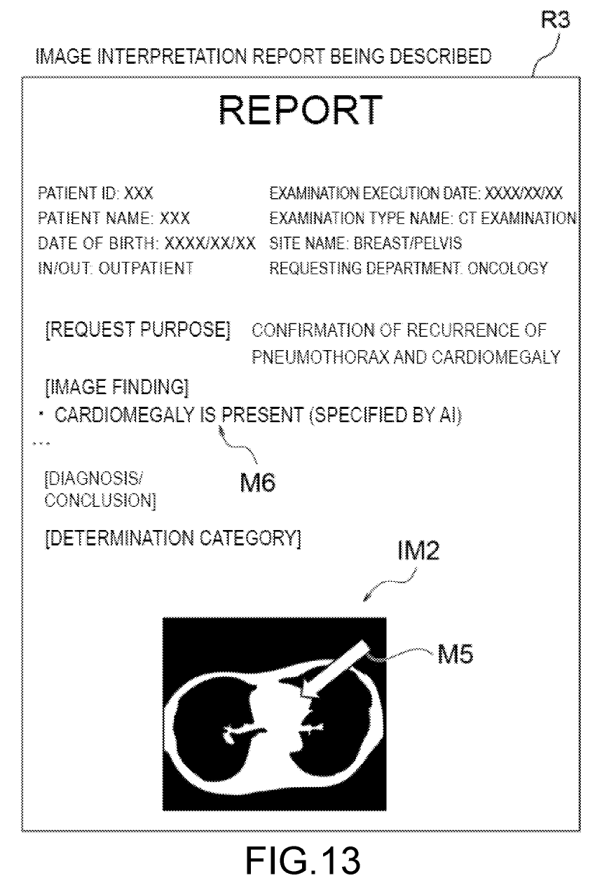

FIG. 13 is a diagram illustrating details of the new report R3 in FIG. 10. In the example of FIG. 13, in the report R3, patient information, examination order information, "request purpose", "image finding, diagnosis / conclusion", and "determination category" are described, for example. In the example of FIG. 13, the patient information includes descriptions of "patient ID", "patient name", "date of birth", and "in / out: outpatient". The examination order information includes descriptions of "examination execution date", "examination type name: CT examination", "site name: chest / pelvis", and "requesting department: oncology". The "request purpose" includes a description of "Confirmation of recurrence of pneumothorax and cardiomegaly". In addition, "image finding" includes a description of "Cardiomegaly is present".

In the report R3 illustrated in FIG. 13, a medical image IM2, a mark M5, and a mark M6 may be displayed. The medical image IM2 is, for example, another medical image related to the same patient as the medical image IM1. The medical image IM1 may be displayed in the report R3. The mark M5 is displayed together with the medical image IM2, and indicates the region of interest D3 or the region of non-interest D4 on the medical image IM2 by an arrow, for example. For example, the mark M5 indicates, among the region of interest D3 and the region of non-interest D4 on the medical image IM2, a region added to the report R3 by the button B3 illustrated in FIGS. 11 and 12. The mark M6 clarifies that a text is generated by the generative model 41. In this manner, the output control function 155 of the processing circuit 15 causes the mark M6 or the like to be displayed to emphasize a description by the generative model 41 in the report R3. The mark M6 may also be displayed in the reports R1 and R2.

Returning to FIG. 10, a user performs an operation on the report R3 using a button B1 or a button B2. For example, in a case in which a user determines that there is a problem in contents of the report R3, the user selects the button B2. In this case, for example, all contents added to the report R3 by the button B3 illustrated in FIGS. 11 and 12 are deleted. On the other hand, in a case in which a user determines that there is no problem in the contents of the report R3, the user selects the button B1. Accordingly, the report R3 is stored in the storage circuit 14 or the like.

The output control function 155 of the processing circuit 15 may cause an icon such as a button for storing the report R1 regarding the region of non-interest or an icon for storing the report R2 regarding the region of interest to be displayed on the screen W1. The output control function 155 may also cause an icon for generating the report R3 by merging the report R1 and the report R2 in one operation to be displayed. The generation function 154 of the processing circuit 15 may automatically generate the report R3 irrespective of user input.

Through the above steps, the report generation processing in the medical image processing system 100 according to the present embodiment is ended.

As described above, with the medical image processing system 100 and the medical image processing apparatus 1 according to the first embodiment, the report R1 regarding the region of non-interest D4 is generated by using the generative model 41, so that overlooking of findings including incidental findings in the region of non-interest D4 can be prevented. In addition, the report R1 is automatically generated by the generative model 41, so that a preparation time for an image interpretation report can be shortened. That is, according to the present embodiment, it is possible to achieve support for creating an image interpretation report capable of preventing overlooking of incidental findings. Therefore, according to the present embodiment, it is possible to reduce medical errors while reducing a burden on a physician.

Furthermore, with the medical image processing system 100 and the medical image processing apparatus 1 according to the present embodiment, the report R2 regarding the region of interest D3 is automatically generated by the generative model 41. Accordingly, overlooking of findings in the region of interest D3 can also be prevented. Furthermore, a user can easily create the new report R3 combining the report R1 and the report R2. Accordingly, a preparation time for an image interpretation report can be further shortened.

(Modification of first embodiment)

In the first embodiment described above, when the specification function 153 of the processing circuit 15 specifies the region of non-interest D4, the "finding filtering" illustrated in FIG. 10 may be added. In the example of FIG. 10, "finding filtering" includes "high incidence" related to findings for which high incidence is expected and "user preference" related to a user's interest. The following describes a modification of the first embodiment to which finding filtering is added focusing on differences from the first embodiment.

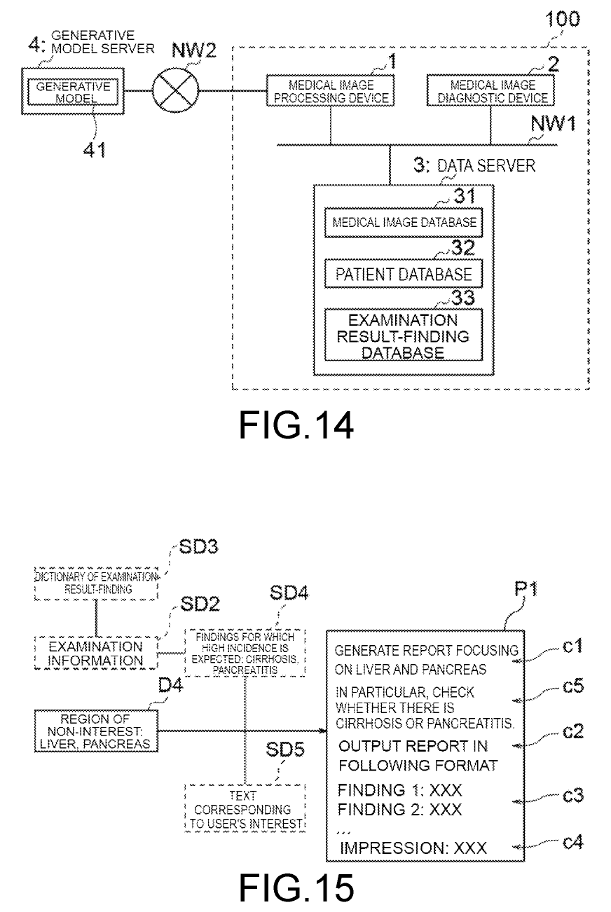

FIG. 14 is a block diagram illustrating a configuration example of the medical image processing system 100 according to the modification of the first embodiment. As illustrated in FIG. 14, the data server 3 of the medical image processing system 100 according to the present modification includes an examination result-finding database 33 in addition to the configuration of the data server 3 of the medical image processing system 100 according to the first embodiment.

The examination result-finding database 33 is a database representing a relation between examination results and findings. The examination result-finding database 33 stores, for example, examination results such as results of prior blood tests and genetic tests, and character strings related to findings and organs for which high incidence is expected from the examination information in association with each other. Specifically, the examination result-finding database 33 stores examination information "secondary findings of a prior genome test indicate BRCA1/2 mutation" and a character string of "chest cancer" in association with each other.

In the present modification, the generation function 154 of the medical image processing apparatus 1 generates the report R1 regarding the region of non-interest D4 based on additional information in addition to the region of non-interest D4. The additional information is, for example, examination information about an examination result of a subject or a user input that is input by a user. That is, in the present modification, the generation function 154 generates the report R1 regarding the region of non-interest D4 based on at least one of the examination information about an examination result of a subject and the user input in addition to the region of non-interest D4. More specifically, in a case in which "high incidence" is selected in "finding filtering" on the screen W1 illustrated in FIG. 10, the generation function 154 generates the report R1 based on the examination information in addition to the region of non-interest D4. In a case in which "user preference" is selected, the generation function 154 generates the report R1 based on a user input in addition to the region of non-interest D4. In a case in which "high incidence" and "user preference" are selected, the generation function 154 generates the report R1 based on both the examination information and the user input in addition to the region of non-interest D4.

The following describes a step of generating a prompt in the present modification with reference to FIG. 15. FIG. 15 is a diagram schematically illustrating an example of a step of generating a prompt related to the region of non-interest in the medical image processing system 100 according to the modification of the first embodiment.

As illustrated in FIG. 15, in the present modification, the generation function 154 of the processing circuit 15 generates the prompt P1 related to the region of non-interest D4 based on at least one of examination information SD2 related to an examination result of a subject and a user input SD5 in addition to the region of non-interest D4. The generation function 154 may also generate the prompt P2 related to the region of interest D3 based on at least one of the examination information SD2 and the user input SD5 in addition to the region of interest D3.

After the region of non-interest D4 is specified by the specification function 153 of the processing circuit 15, the generation function 154 of the processing circuit 15 acquires the examination information SD2 related to the examination result of the subject. Thereafter, for example, the generation function 154 of the processing circuit 15 acquires "cirrhosis, pancreatitis" in a text format and the like as information SD4 related to findings for which high incidence is expected from the examination information SD2 based on a dictionary SD3 of examination result-finding included in the examination result-finding database 33 of the data server 3. As another example, the generation function 154 acquires the examination information SD2 indicating that a BRCA1/2 mutation is present in secondary findings of the prior genome test. In this case, the generation function 154 acquires "chest cancer", for example, as the information SD4.

In addition, the generation function 154 of the processing circuit 15 acquires a text corresponding to a user's interest as the user input SD5. The text corresponding to a user's interest is, for example, a character string representing an item that the user desires to confirm separately from the examination purpose in the current image interpretation.

After acquiring the region of non-interest D4 and at least one of the information SD4 related to findings for which high incidence is expected and the user input SD5, the generation function 154 of the processing circuit 15 generates the prompt P1 for generating the report R1 regarding the region of non-interest D4. In the example of FIG. 15, the prompt P1 further includes an instruction content c5. The instruction content c5 is, for example, an instruction to pay attention to cirrhosis and pancreatitis based on the information SD4 related to findings for which high incidence is expected. In a case based on the user input SD5, content input by a user is described in the instruction content c5 as it is,for example.

As described above, according to the modification of the first embodiment, the contents of the reports R1 to R3 can be made to conform to the examination information based on the prior examination information SD2. In addition, based on the user input SD5 as a text corresponding to the user's interest, desired contents can be included in the reports R1 to R3.

In the present modification, the generation function 154 of the processing circuit 15 may filter descriptions of the reports R1 to R3 by at least one of the information SD4 related to findings for which high incidence is expected and the user input SD5. For example, the generation function 154 extracts descriptions of the information SD4 related to findings for which high incidence is expected and the user input SD5 from the generated reports R1 to R3 using the generative model 41. The output control function 155 of the processing circuit 15 emphasizes the extracted descriptions. Accordingly, the user can easily grasp descriptions of the examination information SD2 and the user input SD5.

### Second embodiment

In the first embodiment described above, the setting function 152 of the processing circuit 15 sets the region of interest in a text format based on the site information D2 in a text format. On the other hand, in a second embodiment described below, the setting function 152 sets the region of interest based on the region information in the medical image. The following describes the present embodiment focusing on differences from the first embodiment.

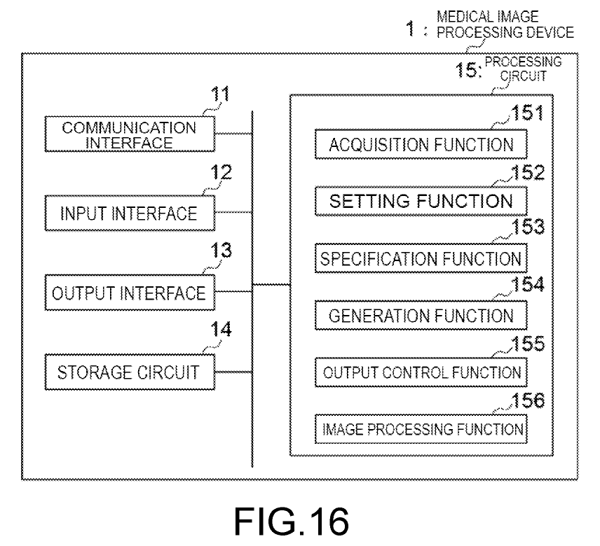

FIG. 16 is a block diagram illustrating a configuration example of the medical image processing apparatus 1 according to the second embodiment. As illustrated in FIG. 16, the processing circuit 15 of the medical image processing apparatus 1 according to the present embodiment further includes an image processing function 156 in addition to the configuration of the processing circuit 15 of the medical image processing apparatus 1 according to the first embodiment.

The image processing function 156 performs image processing on the medical image. In the present embodiment, the image processing function 156 performs predetermined processing, for example, concealment processing, on a processing region that is a partial region in the medical image. The concealment processing is, for example, processing of reducing an amount of information in the processing region by concealing at least part of information included in the processing region, and is processing of performing blacking out, blurring, cutting out, or the like on the processing region. The image processing function 156 corresponds to an image processor in the present embodiment.

In the present embodiment, the acquisition function 151 acquires the region information in the medical image. The region information is, for example, information related to a region surrounding part of the medical image. In the present embodiment, the acquisition function 151 receives an input related to the region information from a user via the input interface 12. For example, the output control function 155 causes the medical image to be displayed on the display, and a user performs an operation of inputting the region information while referring to the medical image. The acquisition function 151 acquires the region information based on the user's operation.

In the present embodiment, the setting function 152 sets the region of interest based on the region information in the medical image acquired by the acquisition function 151. More specifically, the setting function 152 sets the region of interest as the processing region described above based on the region information.

In addition, in the present embodiment, the specification function 153 specifies the region of non-interest by causing the image processing function 156 to perform predetermined processing, for example, the concealment processing, on the processing region set by the setting function 152.

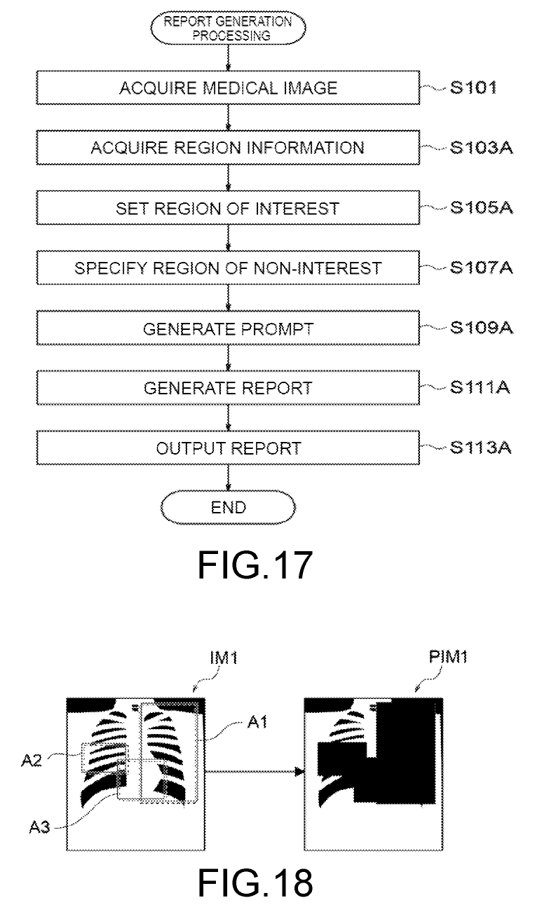

Next, the following describes an example of report generation processing in the medical image processing system 100 according to the present embodiment with reference to FIG. 17. FIG. 17 is a flowchart illustrating an example of the report generation processing in the medical image processing system 100 according to the second embodiment. Since step S101 is the same as that in the first embodiment described above, the description thereof will be omitted.

As illustrated in FIG. 17, after step S101, the acquisition function 151 of the medical image processing apparatus 1 acquires the region information (step S103A). FIG. 18 is a diagram schematically illustrating an example of a process up to a step of specifying the region of non-interest in the medical image processing system according to the second embodiment. As illustrated in FIG. 18, the acquisition function 151 of the processing circuit 15 acquires information about a region A1, a region A2, and a region A3 as the region information in the medical image IM1. The regions A1 to A3 are, for example, sites to which a user pays attention on the medical image IM1 and that are input as the region information. In the example of FIG. 18, the region A1 is a region including an entire left lung, the region A2 is a region including part of a right lung, and the region A3 is a region including an entire heart.

Next, as illustrated in FIG. 17, the setting function 152 of the medical image processing apparatus 1 sets regions of interest (step S105A). More specifically, as illustrated in FIG. 18, the setting function 152 of the processing circuit 15 sets regions of interest based on the region information in the medical image IM1. In the example of FIG. 18, the regions of interest include the region A1, the region A2, and the region A3. The setting function 152 also sets the regions of interest as processing regions in the medical image IM1. In the example of FIG. 18, the processing regions include the region A1, the region A2, and the region A3.

Next, as illustrated in FIG. 17, the specification function 153 of the medical image processing apparatus 1 specifies regions of non-interest (step S107A). As illustrated in FIG. 18, the specification function 153 of the processing circuit 15 causes the image processing function 156 of the processing circuit 15 to perform concealment processing on the processing regions set by the setting function 152 of the processing circuit 15. In the example of FIG. 18, the image processing function 156 performs black-out processing on the regions A1 to A3, which are processing regions, and generates a processed medical image PIM1. In the medical image PIM1, information in the processing regions, that is, the regions corresponding to the regions of interest D3, are concealed by being blacked out. On the other hand, in the medical image PIM1, a remaining portion, that is, a region including the region of non-interest D4, remains without being blacked out. As a result, the region of non-interest D4 in the medical image PIM1 includes a larger amount of information than the region of interest D3 and is emphasized. Therefore, the specification function 153 can specify the region of non-interest D4 by causing the image processing function 156 to generate the medical image PIM1. Thus, in the present embodiment, it can be said that the region of non-interest D4 is a region in the medical image IM1 to which a physician does not normally pay attention when creating an image interpretation report.

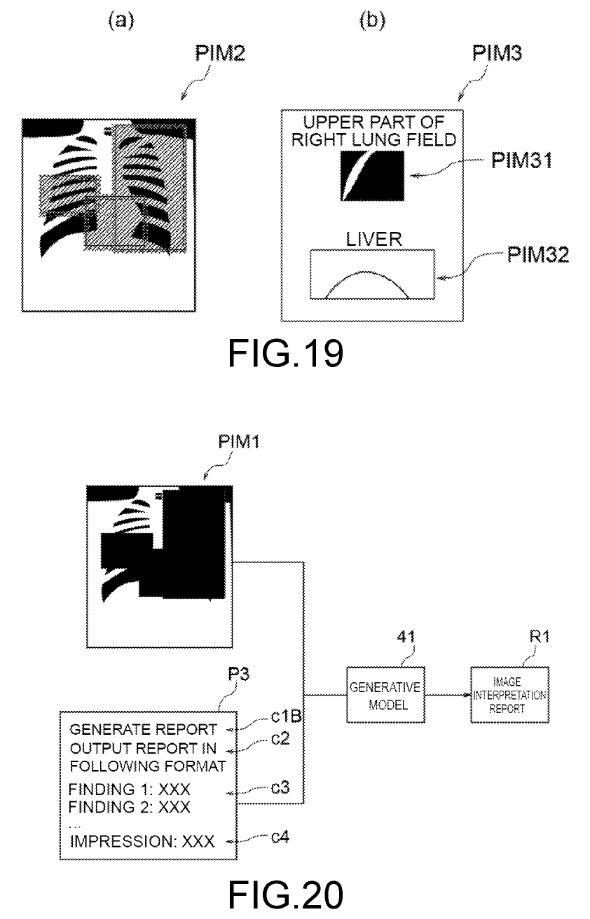

The image processing function 156 of the processing circuit 15 may perform blurring processing or cutting-out processing on the processing region. FIG. 19 is a diagram schematically illustrating another example of a step of specifying the region of non-interest in the medical image processing system 100 according to the second embodiment.

For example, as illustrated in FIG. 19(A), the image processing function 156 of the processing circuit 15 performs blurring processing on the processing region and generates a processed medical image PIM2. In the medical image PIM2, information in the processing regions, that is, the regions A1 to A3 corresponding to the regions of interest D3, is concealed by being blurred. On the other hand, in the medical image PIM2, a remaining portion, that is, the region of non-interest D4 remains without being blurred. As a result, the region of non-interest D4 in the medical image PIM2 is emphasized as compared with the regions of interest D3.

As illustrated in FIG. 19(B), the image processing function 156 of the processing circuit 15 performs cutting-out processing on the processing region and generates the processed medical image PIM3. In the medical image PIM3, information in the processing regions, that is, the regions A1 to A3 corresponding to the regions of interest D3, is concealed by being excluded by the cutting-out processing. On the other hand, in the medical image PIM3, a remaining portion, that is, the region of non-interest D4, remains without being cut out. As a result, the region of non-interest D4 in the medical image PIM3 is emphasized as compared with the regions of interest D3. In the example of FIG. 19(B), the medical image PIM3 includes a medical image PIM31 related to an upper part of a right lung field in the region of non-interest D4 and a medical image PIM32 related to a liver in the region of non-interest D4.

Next, as illustrated in FIG. 17, the generation function 154 of the medical image processing apparatus 1 generates a prompt (step S109A). FIG. 20 is a diagram schematically illustrating an example of a step of generating a prompt in the medical image processing system 100 according to the second embodiment and a step of generating a report regarding the region of non-interest.

As illustrated in FIG. 20, the generation function 154 of the processing circuit 15 generates a prompt P3. Herein, the prompt P3 includes, for example, instruction contents c1B and c2 to c4. The instruction content c1B is an instruction to generate a report. Unlike the instruction content c1 in the first embodiment described above, the instruction content c1B does not include information about the region of non-interest D4. This is because the region of non-interest D4 has been already specified in the processed medical image PIM1. The instruction content c1B may include information of the region of non-interest D4. On the other hand, the instruction contents c2 to c4 are the same as those in the prompt P1 described above.

Next, as illustrated in FIG. 17, the generation function 154 of the medical image processing apparatus 1 generates a report (step S111A). As illustrated in FIG. 20, first, the generation function 154 of the processing circuit 15 transmits and inputs the prompt P3 to the generative model 41 of the generative model server 4 via the communication interface 11. In the present embodiment, the processed medical image PIM1 is input simultaneously with the prompt P3. Accordingly, an instruction is input to the generative model 41 to create the report R1 regarding the region of non-interest D4 in the medical image IM1 based on the prompt P3 and the processed medical image PIM1. Thereafter, the generation function 154 receives an output from the generative model 41 of the generative model server 4 via the communication interface 11. Subsequently, the generation function 154 generates the report R1 based on the received output from the generative model 41.

At steps S109A to S111A described above, the medical image processing apparatus 1 may generate the report R2 regarding the region of interest. In this case, first, the image processing function 156 of the processing circuit 15 generates, for example, an inverted medical image obtained by inverting a blacked-out portion in the processed medical image PIM1. Subsequently, the generation function 154 of the processing circuit 15 transmits and inputs the inverted medical image to the generative model 41 together with the prompt P3. Accordingly, an instruction is input to the generative model 41 to create the report R2 regarding the region of interest D3 in the medical image IM1. Thereafter, the generation function 154 receives an output from the generative model 41 of the generative model server 4 via the communication interface 11. Subsequently, the generation function 154 generates the report R2 based on the received output from the generative model 41.

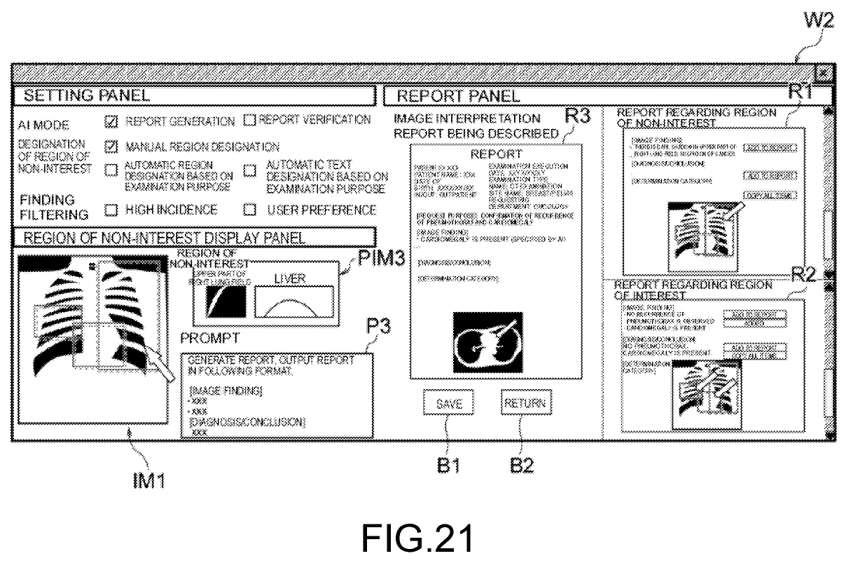

Next, as illustrated in FIG. 17, the output control function 155 of the medical image processing apparatus 1 outputs a report (step S113A). FIG. 21 is a diagram illustrating an example of a report output screen in the medical image processing system 100 according to the second embodiment. The output control function 155 of the processing circuit 15 causes, for example, a screen W2 illustrated in FIG. 21 to be output to the display. The following describes the screen W2 focusing on differences from the screen W1 in FIG. 10.

As illustrated in FIG. 21, on the screen W2, "manual region designation" is selected as "designation of region of non-interest" in the "setting panel", and an operation mode is selected in which the specification function 153 of the processing circuit 15 specifies the region of non-interest from the region information designated by a user.

In addition, on the "region of non-interest display panel", the medical image IM1, the processed medical image PIM3, and the prompt P3 are displayed. Accordingly, a user can intuitively grasp the region of interest and the region of non-interest, for example. The user may be allowed to directly edit the region information included in the medical image IM1 or contents of the prompt P3, for example.

Through the above steps, the report generation processing in the medical image processing system 100 according to the present embodiment is ended.

As described above, with the medical image processing system 100 and the medical image processing apparatus 1 according to the second embodiment, the report R1 regarding the region of non-interest can be automatically generated based on the region information designated by the user. In addition, the user can intuitively set the region of interest while viewing the medical image.

### Modification of Second Embodiment

In the second embodiment described above, the acquisition function 151 of the processing circuit 15 acquires the region information based on the user input. On the other hand, in a modification of the second embodiment described below, the acquisition function 151 automatically generates and acquires the region information based on the medical image and the site information. It can be said that the present modification is obtained by merging functions of the first embodiment and the second embodiment. The following describes the present modification focusing on differences from the second embodiment.

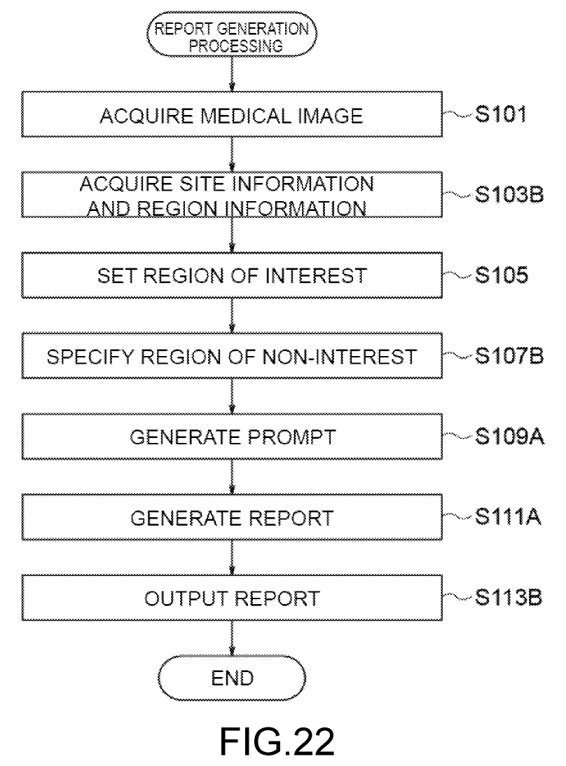

FIG. 22 is a flowchart illustrating an example of report generation processing in the medical image processing system 100 according to the modification of the second embodiment. Since step S101 is the same as that in the first embodiment described above, the description thereof will be omitted.

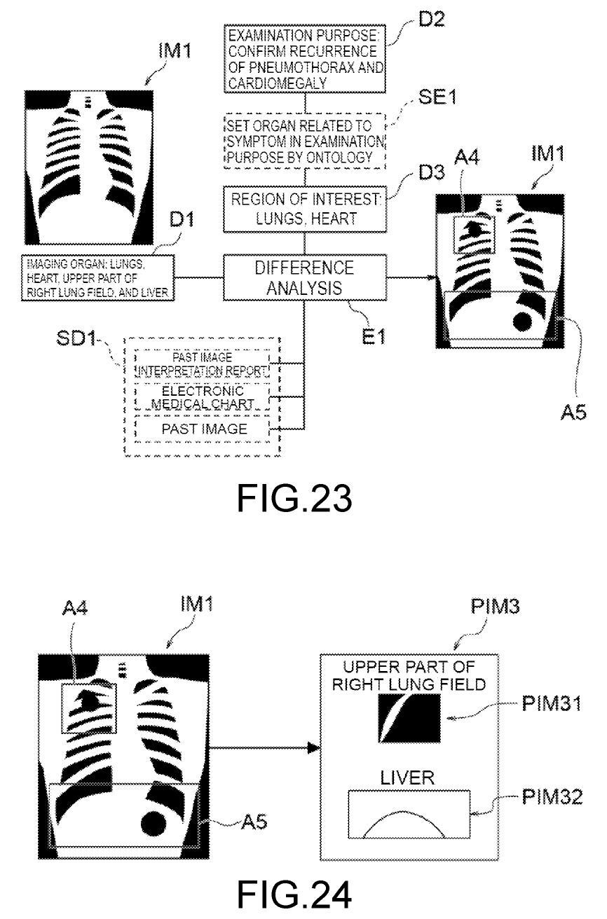

As illustrated in FIG. 22, after step S101, the acquisition function 151 of the medical image processing apparatus 1 acquires the site information and the region information (step S103B). FIG. 23 is a diagram schematically illustrating an example of a process up to a step of specifying the region of non-interest in the medical image processing system 100 according to the modification of the second embodiment.

As illustrated in FIG. 23, in the present embodiment, the acquisition function 151 of the processing circuit 15 acquires the site information D2 related to the examination purpose of the subject. The acquisition function 151 also acquires the imaging target information D1 of the medical image IM1. In the example of FIG. 23, the imaging target information D1 of the medical image IM1 includes lungs, a heart, and the like. In the present modification, the acquisition function 151 acquires the imaging target information D1 using a learning model for detecting a site included in the medical image IM1 (hereinafter, also referred to as a "site detection model"), and acquires the region information corresponding to the imaging target information D1 at the same time. Therefore, the acquisition function 151 acquires both the site information D2 related to the examination purpose of the subject and the region information in the medical image IM1. The acquisition function 151 may also acquire the imaging target information D1 and the region information corresponding to the imaging target information D1 from the medical image IM1 by using a segmentation model. Since the next step S105 is the same as that in the first embodiment described above, the description thereof will be omitted. However, in the present modification, the region of interest D3 set by the setting function 152 of the processing circuit 15 corresponds to both the site information D2 and the region information.

As illustrated in FIG. 22, after step S105, the specification function 153 of the medical image processing apparatus 1 specifies the region of non-interest (step S107B). More specifically, as illustrated in FIG. 23, the specification function 153 of the processing circuit 15 specifies the region of non-interest by the difference analysis E1 between the imaging target information D1 and the region of interest D3. In the example of FIG. 23, the region of non-interest includes, for example, "upper part of right lung field" and a "liver" as sites not included in the region of interest D3 among the sites included in the imaging target information D1. The specification function 153 also specifies the region of non-interest as a region in the medical image IM1. In the example of FIG. 23, the specification function 153 specifies a region A4 corresponding to the "upper part of right lung field" and a region A5 corresponding to the "liver". Thus, in the present modification, it can be said that the region of non-interest D4 is a character string representing a finding or a site that is unlikely to be assumed from the examination purpose and the like included in the site information D2, and is a region in the medical image IM1 corresponding to the character string.

FIG. 24 is a diagram schematically illustrating an example of a process up to a step of specifying the region of non-interest in the medical image processing system 100 according to the modification of the second embodiment subsequent to FIG. 23. As illustrated in FIG. 24, after specifying the regions A4 and A5, the specification function 153 of the processing circuit 15 causes the image processing function 156 of the processing circuit 15 to process the medical image IM1 based on the regions A4 and A5. In the present modification, cutting-out processing is performed on regions other than the regions A4 and A5, and the processed medical image PIM3 is generated. The medical image PIM3 includes the medical image PIM31 regarding the upper part of the right lung field and the medical image PIM32 regarding the liver. Alternatively, the specification function 153 of the processing circuit 15 may cause the image processing function 156 of the processing circuit 15 to process regions corresponding to sites included in the region of interest D3 in place of the regions other than the regions A4 and A5.

The image processing function 156 of the processing circuit 15 may generate the processed medical image PIM1 illustrated in FIG. 18 by performing black-out processing on the regions other than the regions A4 and A5, or may generate the processed medical image PIM2 illustrated in FIG. 19(A) by performing blurring processing thereon.

In the example described above, at step S103B, the acquisition function 151 acquires the region information related to the imaging target information D1 from the medical image IM1 by using the site detection model. The embodiment is not limited thereto, and the acquisition function 151 may acquire the region information related to the region of non-interest D4 from the medical image IM1 by using the site detection model at step S107B. That is, the acquisition function 151 may perform site detection for sites not included in the region of interest D3 in the medical image IM1. Since steps S109A and S111A are the same as those in the second embodiment described above, the description thereof will be omitted.

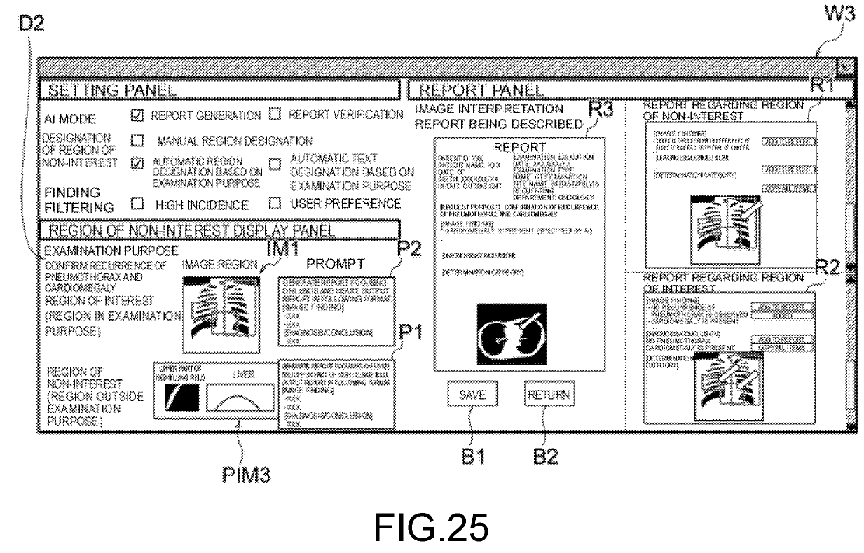

After step S111A, as illustrated in FIG. 17, the output control function 155 of the medical image processing apparatus 1 outputs a report (step S113B). FIG. 25 is a diagram illustrating an example of the report output screen in the medical image processing system 100 according to the modification of the second embodiment. The output control function 155 of the processing circuit 15 causes, for example, a screen W3 illustrated in FIG. 25 to be output to the display. The following describes the screen W3 focusing on differences from the screen W2 in FIG. 21.

As illustrated in FIG. 25, on the screen W3, "automatic region designation based on examination purpose" is selected as "designation of region of non-interest" in "setting panel", and an operation mode is selected in which the specification function 153 of the processing circuit 15 specifies the region of non-interest from the region information that is automatically acquired from the site information D2 including the examination purpose.

In addition, in "region of non-interest display panel", the medical image IM1, the processed medical image PIM3, the site information D2, the prompt P1, and the prompt P2 are displayed.

Through the above steps, the report generation processing in the medical image processing system 100 according to the present modification is ended.

As described above, with the medical image processing system 100 and the medical image processing apparatus 1 according to the modification of the second embodiment, the region information can be automatically acquired and a burden on a physician can be reduced as compared with the second embodiment.

In the above description, the medical image processing system 100 and the medical image processing apparatus 1 according to the present modification are configured by merging the functions of the first embodiment and the second embodiment. The embodiment is not limited thereto, and the medical image processing system 100 and the medical image processing apparatus 1 according to the present modification may be configured by merging the functions of the modification of the first embodiment and the second embodiment. For example, the acquisition function 151 may acquire the information SD4 related to findings for which high incidence is expected and the region information related to the user input SD5 from the medical image IM1 by using the site detection model.

### Third Embodiment

In the embodiments described above, the reports R1 to R3 as image interpretation reports are generated from the medical image IM1. On the other hand, the medical image processing system 100 according to a third embodiment described below has a function of verifying an image interpretation report in a case in which the image interpretation report regarding the medical image IM1 has been already created. The following describes the present embodiment focusing on differences from the modification of the second embodiment.

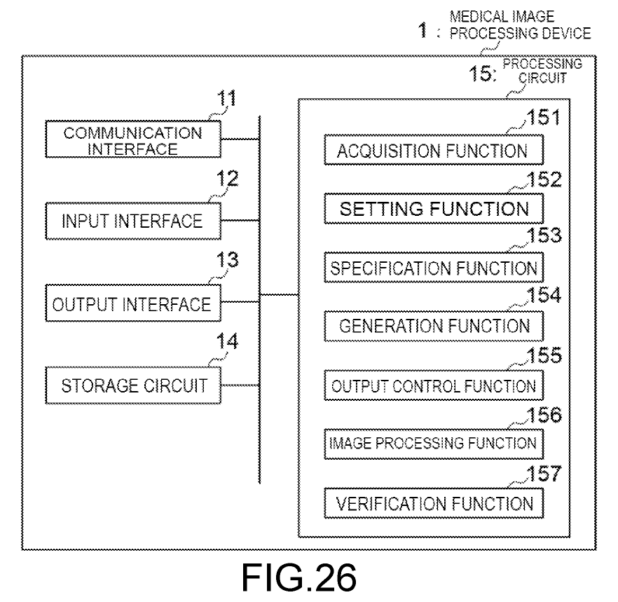

FIG. 26 is a block diagram illustrating a configuration example of the medical image processing apparatus 1 according to the third embodiment. As illustrated in FIG. 26, the processing circuit 15 of the medical image processing apparatus 1 according to the present embodiment has a verification function 157 in addition to the configuration of the processing circuit 15 of the medical image processing apparatus 1 according to the second embodiment illustrated in FIG. 16.

The verification function 157 verifies an interpreted image interpretation report that has already been interpreted by a physician or the like. For example, the verification function 157 verifies the interpreted image interpretation report of the medical image IM1 that has already been interpreted based on the report R1 regarding the region of non-interest D4 generated by the generation function 154 for the medical image IM1. Specifically, the verification function 157 generates a verification report regarding the region of non-interest D4 based on the report R1 and the interpreted image interpretation report. The verification function 157 may verify the interpreted image interpretation report of the medical image IM1 that has already been interpreted based on the report R2 regarding the region of interest D3 generated by the generation function 154 for the medical image IM1, or may verify the interpreted image interpretation report of the medical image IM1 that has already been interpreted based on the new report R3. The verification function 157 corresponds to a verifier in the present embodiment.

In the present embodiment, the output control function 155 causes the output interface 13 to output the report verified by the verifier.

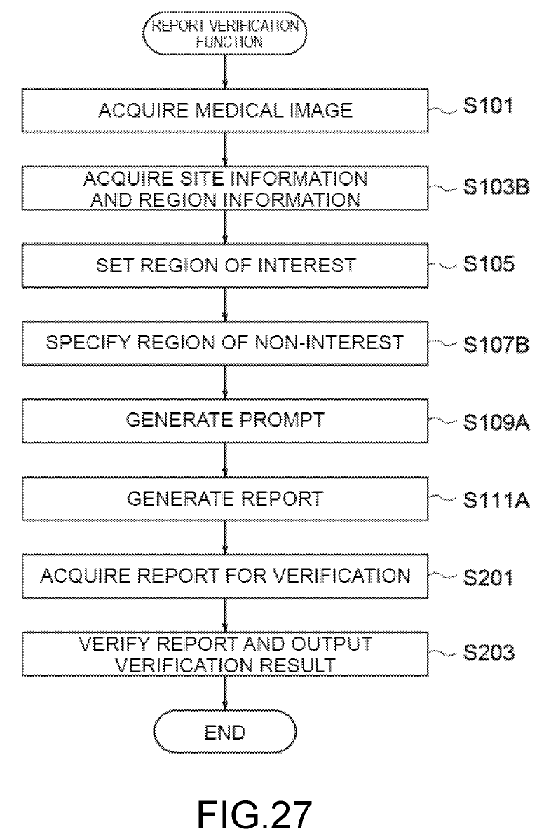

Next, with reference to FIG. 27, the following describes an example of report verification processing in the medical image processing system 100 according to the present embodiment. FIG. 27 is a flowchart illustrating an example of the report verification processing in the medical image processing system 100 according to the third embodiment. The report verification processing is performed, for example, when the user verifies the interpreted image interpretation report by using the medical image processing system 100. In the report verification processing illustrated in FIG. 27, steps S101 to S111A are the same as those in the report generation processing in the modification of the second embodiment illustrated in FIG. 22, so that the description thereof will be omitted.

After step S111A, as illustrated in FIG. 27, the acquisition function 151 of the medical image processing apparatus 1 acquires a report for verification (step S201). More specifically, the acquisition function 151 of the processing circuit 15 acquires an interpreted image interpretation report for verification from the data server 3 or the like.

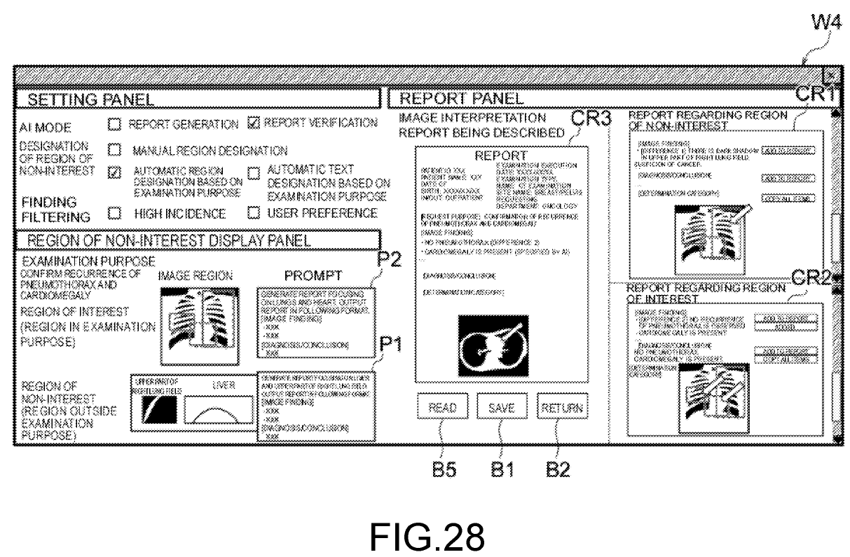

Next, as illustrated in FIG. 27, the verification function 157 of the medical image processing apparatus 1 verifies the report, and the output control function 155 of the medical image processing apparatus 1 outputs a verification result (step S203). FIG. 28 is a diagram illustrating an example of a report verification screen in the medical image processing system 100 according to the third embodiment. The output control function 155 of the processing circuit 15 causes, for example, a screen W4 illustrated in FIG. 28 to be output to the display. The following describes the screen W4 focusing on differences from the screen W3 in FIG. 25.

As illustrated in FIG. 28, on the screen W4, "report generation" is selected as "AI mode", and an operation mode of performing report verification processing illustrated in FIG. 27 is selected.

In addition, on the screen W4, a button B5 is displayed in "report panel". The user inputs an instruction to acquire an image interpretation report for verification by the button B5. For example, when the user presses the button B5, the acquisition function 151 of the processing circuit 15 acquires the interpreted image interpretation report for verification from the data server 3 or the like.

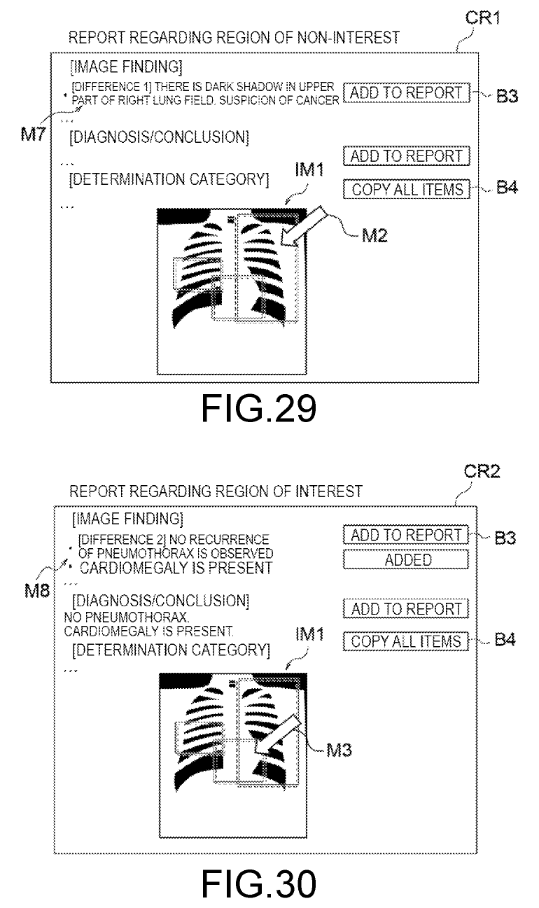

In "report panel", verification reports CR1 to CR3 are displayed. Each of the verification reports CR1 to CR3 is an example of a verification result of the image interpretation report by the verification function 157 of the medical image processing apparatus 1. The following describes details of the verification reports CR1 to CR3 focusing on differences from the reports R1 to R3 illustrated in FIGS. 11-13, respectively, with reference to FIGS. 29-31.

FIG. 29 is a diagram illustrating details of the verification report CR1 regarding the region of non-interest in FIG. 28. As illustrated in FIG. 29, the verification function 157 of the processing circuit 15 verifies the acquired interpreted image interpretation report based on the report R1 regarding the region of non-interest D4 generated by the generation function 154 of the processing circuit 15, and generates the verification report CR1. In the example of FIG. 29, a mark M7 is displayed in the verification report CR1. The mark M7 represents a difference between the report R1 and the interpreted image interpretation report as "difference 1". In this manner, the output control function 155 of the processing circuit 15 displays the mark M7 or the like, thereby emphasizing a difference between the report R1 generated by the generation function 154 and a verification result of the interpreted image interpretation report by the verification function 157. Accordingly, the user can easily grasp a difference between the report R1 and the interpreted image interpretation report.

FIG. 30 is a diagram illustrating details of a verification report CR2 regarding the region of interest in FIG. 28. As illustrated in FIG. 30, the verification function 157 of the processing circuit 15 verifies the acquired interpreted image interpretation report based on the report R2 regarding the region of interest D3 generated by the generation function 154 of the processing circuit 15, and generates the verification report CR2. A mark M8 is displayed in the verification report CR2. Similarly to the mark M7, the mark M8 represents a difference between the report R2 and the interpreted image interpretation report as "difference 2". Accordingly, the user can easily grasp a difference between the report R2 and the interpreted image interpretation report.

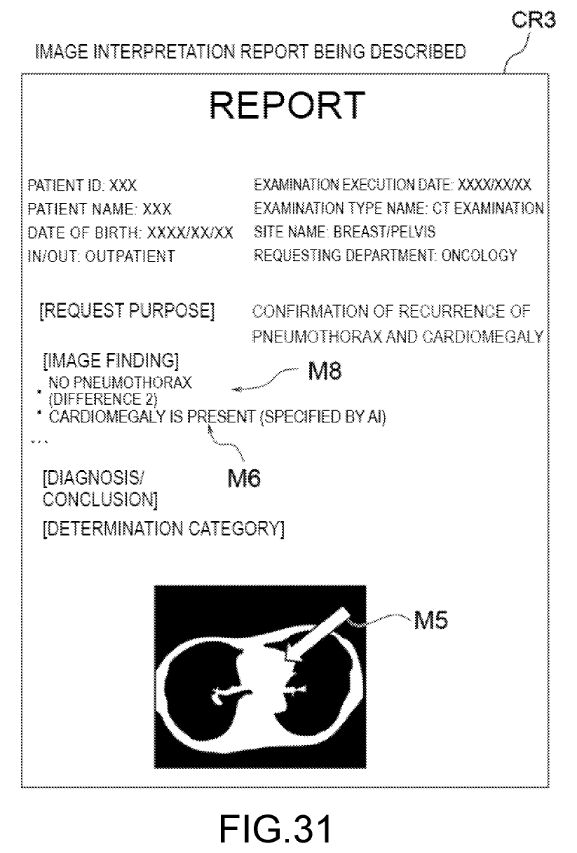

FIG. 31 is a diagram illustrating details of the new verification report CR3 in FIG. 28. As illustrated in FIG. 31, the verification function 157 of the processing circuit 15 verifies the acquired interpreted image interpretation report based on the new report R3 generated by the generation function 154 of the processing circuit 15, and generates the verification report CR3. In the verification report CR3, for example, the mark M8 is displayed similarly to FIG. 30.

As described above, with the medical image processing system 100 and the medical image processing apparatus 1 according to the third embodiment, by generating the verification report CR1 regarding the region of non-interest D4, overlooking of incidental findings in the image interpretation report can be prevented. In addition, by generating the verification report CR1, it is possible to more easily confirm validity of a finding included in a report interpreted by a physician for the region of non-interest D4. Furthermore, by generating the verification report CR2 regarding the region of interest D3 or the verification report CR3 regarding the new report R3, it is possible to more easily confirm validity of findings included in a report interpreted by a physician, for example, findings other than incidental findings, for the region of interest D3.

### Fourth Embodiment

In the embodiments described above, the generative model 41 is provided in the generative model server 4. On the other hand, in a fourth embodiment described below, the generative model 41 is provided in the medical image processing apparatus 1. The following describes the present embodiment focusing on differences from the first embodiment.

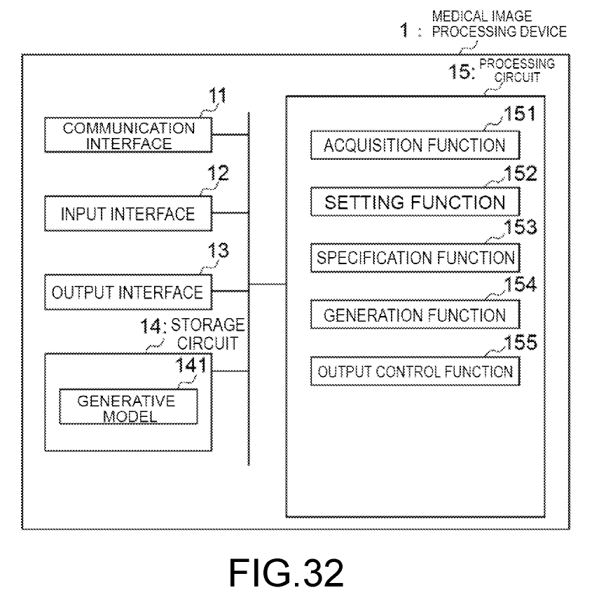

FIG. 32 is a block diagram illustrating a configuration example of the medical image processing apparatus 1 according to the fourth embodiment. As illustrated in FIG. 32, the medical image processing apparatus 1 according to the present embodiment includes a generative model 141 in addition to the configuration of the medical image processing apparatus 1 of the embodiment. The generative model 141 corresponds to the generative model 41 in the first embodiment described above, and has a substantially equivalent function. In the present embodiment, the communication network NW2 in FIG. 1 may be omitted.

In the present embodiment, the generation function 154 inputs a prompt to the generative model 141. Subsequently, the generation function 154 acquires a report output from the generative model 141.

As described above, with the medical image processing system 100 and the medical image processing apparatus 1 according to the fourth embodiment, it is possible to achieve support for creating an image interpretation report capable of preventing overlooking of incidental findings even in a so-called on-premise generative model 141. The medical image processing apparatus 1 according to the present embodiment can also be used in an environment without a communication connection with an external network. Accordingly, for example, security of the medical image processing system 100 can be improved.

### FIFTH EMBODIMENT

Next, the following describes the medical image processing system 100 according to a fifth embodiment including an X-ray imaging apparatus 5 as the medical image diagnostic apparatus 2 in the first embodiment. The following describes the present embodiment focusing on differences from the first embodiment.

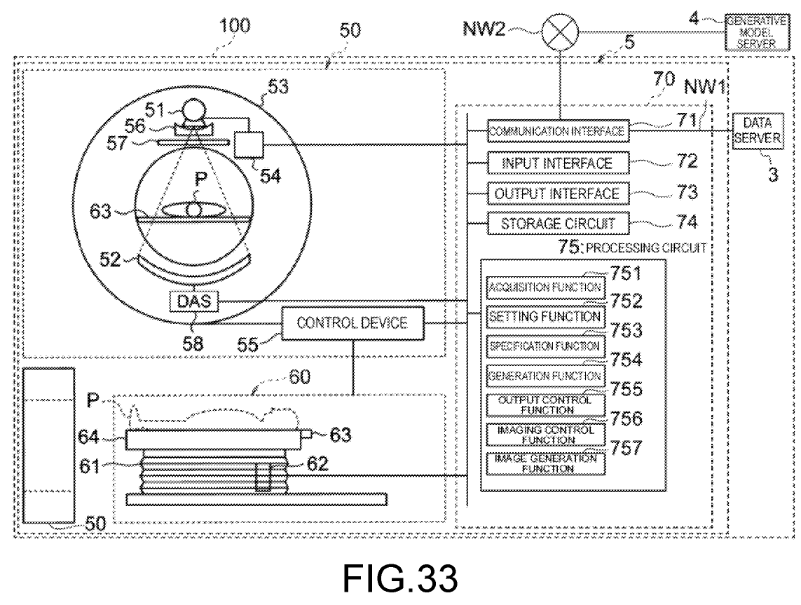

FIG. 33 is a block diagram illustrating a configuration example of the medical image processing system 100 according to the fifth embodiment. As illustrated in FIG. 33, the medical image processing system 100 according to the present embodiment includes the X-ray imaging apparatus 5 as one of the medical image diagnostic apparatus 2. In the present embodiment, the medical image processing apparatus 1 is incorporated in the X-ray imaging apparatus 5 as a console device 70. The following description exemplifies a case in which the X-ray imaging apparatus 5 is an X-ray CT apparatus. The X-ray imaging apparatus 5 may be an X-ray imaging apparatus other than the X-ray CT apparatus such as a general X-ray diagnostic apparatus.

The X-ray imaging apparatus 5 images a subject P using X-rays and generates an X-ray CT image. The X-ray imaging apparatus 5 includes, for example, a platform device 50, a couch device 60, and the console device 70. For convenience of explanation, a plurality of platform devices 50 are depicted in FIG. 33, but actually, one platform device 50 is provided.

The platform device 50 includes an X-ray tube 51, an X-ray detector 52, a rotary frame 53, an X-ray high voltage device 54, a control device 55, a wedge 56, a collimator 57, and a Data Acquisition System (DAS) 58.

The X-ray tube 51 is a vacuum tube that generates X-rays by emitting thermoelectrons from a cathode (filament) toward an anode (target) with a high voltage from the X-ray high voltage device 54. The present embodiment is applicable to both a single-tube type X-ray CT apparatus and a so-called multi-tube type X-ray CT apparatus in which a plurality of pairs of X-ray tubes and detectors are mounted on a rotary ring. Hardware that generates X-rays is not limited to the X-ray tube 51. For example, instead of the X-ray tube 51, X-rays may be generated by using a fifth-generation method including a focus coil that focuses an electron beam generated from an electron gun, a deflection coil that electromagnetically deflects the electron beam, and a target ring that surrounds a half circumference of the subject P and generates X-rays by collision of the deflected electron beam.

The X-ray high voltage device 54 includes a high voltage generation circuit that includes an electrical circuit such as a transformer (trans) and a rectifier and generates a high voltage to be applied to the X-ray tube 51, and an X-ray control circuit that controls an output voltage corresponding to X-rays emitted by the X-ray tube 51. The high voltage generation circuit may be a transformer system or an inverter system. The X-ray high voltage device 54 not only generates a high voltage but also supplies power to the filament and supplies drive power when the anode is of a rotary type. The X-ray high voltage device 54 may be provided on the rotary frame 53, or may be provided on a fixed frame side of the platform device 50. The fixed frame is a frame that supports the rotary frame 53 in a rotatable manner.

The X-ray detector 52 detects X-rays emitted from the X-ray tube 51 and passed through the subject P, and outputs a signal corresponding to an amount of detected X-rays to the DAS 58. The X-ray detector 52 includes, for example, a plurality of X-ray detection element arrays in which a plurality of X-ray detection elements are arranged in a channel direction (circumferential direction) along one arc centered on a focal point of the X-ray tube 51. The X-ray detector 52 has, for example, a structure in which a plurality of X-ray detection element arrays, each including a plurality of X-ray detection elements arranged in the channel direction, are arranged in a slice direction (a column direction, a row direction).

The X-ray detector 52 is, for example, a detector of an indirect conversion type including a grid, a scintillator array, and an optical sensor array. The scintillator array includes a plurality of scintillators. The scintillator includes a scintillator crystal that outputs light having a photon amount corresponding to an amount of incident X-rays. The grid is arranged on a surface on an X-ray incident side of the scintillator array, and includes an X-ray shielding plate that absorbs scattered X-rays. The grid may also be referred to as a collimator (a one-dimensional collimator or a two-dimensional collimator). The optical sensor array has a function of converting light from the scintillator into an electric signal corresponding to an amount of the light and includes, for example, an optical sensor such as a photodiode or a photomultiplier (PMT). The X-ray detector 52 may be a detector of a direct conversion type including a semiconductor element that converts incident X-rays into an electric signal.

The rotary frame 53 (platform base) is an annular frame that supports the X-ray tube 51 and the X-ray detector 52 to face each other, and rotates the X-ray tube 51 and the X-ray detector 52 by the control device 55. For example, the rotary frame 53 is a casting made of aluminum. The rotary frame 53 may further support the X-ray high voltage device 54 and the DAS 58 in addition to the X-ray tube 51 and the X-ray detector 52. Furthermore, the rotary frame 53 can further support various configurations not illustrated in FIG. 33. Hereinafter, in the platform device 50, a portion that rotates and moves together with the rotary frame 53 and the rotary frame 53 may also be referred to as a rotary portion. Although a Rotate/Rotate-Type (third-generation CT), in which the X-ray tube 51 and the X-ray detector 52 integrally rotate around the subject P, has been described, there are also various other types such as a Stationary/Rotate-Type (fourth-generation CT), in which a large number of X-ray detection elements arrayed in a ring shape are fixed and only the X-ray tube 51 rotates around the subject P, and any of these types is applicable to the present embodiment.

Detection data generated by the DAS 58 is transmitted from a transmitter including a Light Emitting Diode (LED) provided on the rotary frame 53 to a receiver including a photodiode provided on a non-rotary portion of the platform device 50 by optical communication, and transferred to the console device 70. Herein, the non-rotary portion is, for example, a fixed frame or the like that supports the rotary frame 53 in a rotatable manner. A method for transmitting the detection data from the rotary frame 53 to the non-rotary portion of the platform device 50 is not limited to the optical communication, and any scheme capable of performing data transmission between the rotary portion and the non-rotary portion may be employed.

The control device 55 includes a drive mechanism such as a motor and an actuator, and a circuit that controls the mechanism. The control device 55 receives an input signal from an input interface 72, an input interface provided in the platform device 50, or the like, and performs operation control of the platform device 50 and the couch device 60. For example, the control device 55 controls rotation of the rotary frame 53, a tilt of the platform device 50, an operation of the couch device 60, and the like. By way of example, the control device 55 rotates the rotary frame 53 about an axis parallel to an X-axis direction based on input inclination angle (tilt angle) information as control for tilting the platform device 50. The control device 55 may be provided in the platform device 50, or may be provided in the console device 70.

The wedge 56 is a filter for adjusting an amount of X-rays emitted from the X-ray tube 51. Specifically, the wedge 56 is a filter that transmits and attenuates X-rays emitted from the X-ray tube 51 so that X-rays emitted from the X-ray tube 51 to the subject P have a predetermined distribution. For example, the wedge 56 is a wedge filter or a bow-tie filter, and is configured by processing aluminum or the like to have a predetermined target angle and a predetermined thickness.

The collimator 57 is a lead plate or the like for narrowing down an irradiation range of X-rays transmitted through the wedge 56, and forms a slit by a combination of a plurality of lead plates or the like. The collimator 57 may also be referred to as an X-ray aperture. An opening degree and a position of the collimator 57 are adjusted by a collimator adjustment circuit which is not illustrated in the drawings. Accordingly, the irradiation range of X-rays generated by the X-ray tube 51 is adjusted.

The DAS 58 includes an amplifier that performs amplification processing on an electric signal output from each X-ray detection element of the X-ray detector 52, and an A/D converter that converts the electric signal into a digital signal, and generates detection data. The DAS 58 is, for example, implemented by a processor. The detection data generated by the DAS 58 is transferred to the console device 70.

The couch device 60 is a device on which the subject P as a scan target is placed and moved. The subject P is, for example, a human patient or an animal. The couch device 60 includes a base 61, a couch drive device 62, a top plate 63, and a support frame 64. The base 61 is a housing that supports the support frame 64 to be movable in a vertical direction. The couch drive device 62 is a drive mechanism that moves the top plate 63 on which the subject P is placed along a major axis direction of the top plate 63. The top plate 63 is a plate on which the subject P is placed, and is arranged on an upper surface of the support frame 64. In the couch device 60 of the present embodiment, when the top plate 63 is moved along the major axis direction of the top plate 63, the subject P placed on the top plate 63 can be moved between an imaging position at which imaging is performed by the platform device 50 and a retracted position retracted from the platform device 50.

The console device 70 has a function substantially equivalent to that of the medical image processing apparatus 1 in the first embodiment described above. The console device 70 includes, for example, a communication interface 71, an input interface 72, an output interface 73, a storage circuit 74, and a processing circuit 75. The communication interface 71, the input interface 72, the output interface 73, the storage circuit 74, and the processing circuit 75 respectively correspond to the communication interface 11, the input interface 12, the output interface 13, the storage circuit 14, and the processing circuit 15 in the first embodiment described above, and have substantially equivalent functions.

The processing circuit 75 includes an acquisition function 751, a setting function 752, a specification function 753, a generation function 754, and an output control function 755. The acquisition function 751, the setting function 752, the specification function 753, the generation function 754, and the output control function 755 respectively correspond to the acquisition function 151, the setting function 152, the specification function 153, the generation function 154, and the output control function 155 in the first embodiment described above, and have substantially equivalent functions.

The processing circuit 75 also includes an imaging control function 756 and an image generation function 757 in addition to the acquisition function 751, the setting function 752, the specification function 753, the generation function 754, and the output control function 755.

The imaging control function 756 images the subject P by controlling the X-ray tube 51 and the X-ray detector 52. More specifically, the imaging control function 756 controls the couch drive device 62 to move the subject P into an imaging port of the platform device 50. The imaging control function 756 also controls the X-ray high voltage device 54 to supply a high voltage to the X-ray tube 51. The imaging control function 756 also adjusts an opening degree and a position of the collimator 57. The imaging control function 756 also controls the control device 55 to rotate a rotary portion including the rotary frame 53. The imaging control function 756 also causes the DAS 58 to collect projection data. To reconstruct a CT image, projection data for one full rotation, that is, 360°, around the subject P is required, and even in a half-scan method, projection data for "180° + fan angle" is required. Any of the reconstruction schemes can be applied to the present embodiment. The imaging control function 756 corresponds to an imaging controller in the present embodiment.

The image generation function 757 generates an X-ray CT image regarding the subject P based on an output from the X-ray detector 52 by imaging performed by the imaging control function 756. In the present embodiment, the image generation function 757 generates, for example, the medical image IM1 regarding the subject P based on an output from the X-ray detector 52 by imaging performed by the imaging control function 756. The image generation function 757 generates data obtained by performing preprocessing such as logarithm conversion processing, offset correction processing, sensitivity correction processing between channels, or beam hardening correction on detection data output from the DAS 58. The data (detection data) before being subjected to the preprocessing and the data after the preprocessing may be collectively referred to as projection data. In addition, for example, the image generation function 757 generates CT image data. Specifically, the image generation function 757 generates CT image data by performing reconstruction processing using a filtered back projection method, a successive approximation reconstruction method, or the like on the projection data after the preprocessing. The image generation function 757 also converts the CT image data into tomogram data of an arbitrary cross section or three-dimensional image data based on an input operation received from an operator via the input interface 72. The image generation function 757 corresponds to an image generator in the present embodiment.

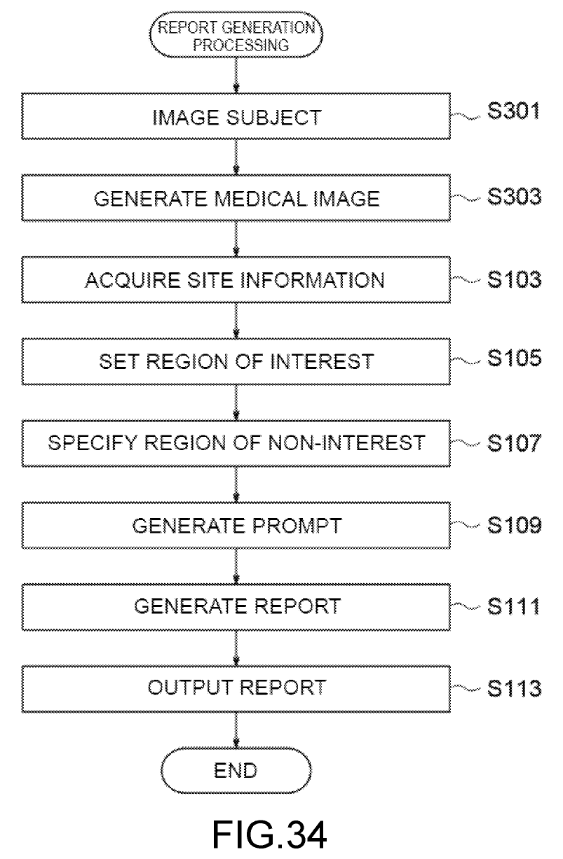

Next, with reference to FIG. 34, the following describes an example of report generation processing in the medical image processing system 100 according to the present embodiment. FIG. 34 is a flowchart illustrating an example of the report generation processing in the medical image processing system 100 according to the fifth embodiment.

First, as illustrated in FIG. 34, the imaging control function 756 of the X-ray imaging apparatus 5 images the subject P (step S301). More specifically, first, the subject P is placed on the top plate 63 of the couch device 60 illustrated in FIG. 33. Thereafter, the imaging control function 756 of the processing circuit 75 moves the top plate 63 with respect to the support frame 64. Accordingly, a site of the subject P to be imaged is inserted into the rotary frame 53. Subsequently, the imaging control function 756 of the processing circuit 75 performs imaging of the subject P.

Next, as illustrated in FIG. 34, the image generation function 757 of the X-ray imaging apparatus 5 generates a medical image (step S303). More specifically, the image generation function 757 of the processing circuit 75 generates a medical image IMG1 of the subject P imaged at step S301. Since steps S103 to S113 are the same as those in the first embodiment described above, the description thereof will be omitted. Through the above steps, the report generation processing in the medical image processing system 100 according to the present embodiment is ended.

As described above, with the medical image processing system 100 according to the fifth embodiment, it is possible to achieve support for creating an image interpretation report capable of preventing overlooking of incidental findings for a medical image imaged by the X-ray imaging apparatus 5.

The term of "processor" in the above description means, for example, a Central Processing Unit (CPU), a Graphics Processing Unit (GPU), or a circuit such as an Application Specific Integrated Circuit (ASIC) and a programmable logic device (for example, a Simple Programmable Logic Device (SPLD), a Complex Programmable Logic Device (CPLD), and a Field Programmable Gate Array (FPGA)). The processor implements a function by reading out and executing a program stored in a storage circuit. Instead of storing a program in the storage circuit, the program may be directly incorporated in a circuit of the processor. In this case, the processor implements a function by reading out and executing the program incorporated in the circuit. The processor is not necessarily configured as a single circuit, but may be configured as one processor by combining a plurality of independent circuits to implement a function thereof. Furthermore, the function thereof may be implemented by integrating a plurality of constituent elements in FIG. 2 into one processor.

According to at least one of the embodiments described above, it is possible to achieve support for creating an image interpretation report capable of preventing overlooking of incidental findings.

Although several embodiments have been described above, these embodiments have been presented only as examples, and are not intended to limit the scope of the invention. The novel devices and methods described herein can be implemented in a variety of other forms. In addition, various omissions, substitutions, and changes can be made to the forms of the apparatus and the method described in the present specification without departing from the gist of the invention. The appended claims and their equivalents are intended to include such forms and modifications as fall within the scope and spirit of the invention.

1. An X-ray imaging apparatus comprising:
an X-ray tube;
an X-ray detector configured to detect X-rays emitted from the X-ray tube and passed through a subject;
an imaging controller configured to perform imaging of the subject by controlling the X-ray tube and the X-ray detector;
an image generator configured to generate a medical image regarding the subject based on an output from the X-ray detector by the imaging;
an acquirer configured to acquire a medical image of the subject and at least one of site information related to an examination purpose of the subject and region information in the medical image;
a setting device configured to set a first region of interest corresponding to at least one of the site information related to the examination purpose of the subject and the region information in the medical image;
a specifier configured to specify a second region of interest other than the first region of interest based on the first region of interest; and
a generator configured to generate a report regarding the second region of interest by using a generative model.

2. A medical image processing apparatus comprising
processing circuitry configured to:
acquire a medical image of a subject, and at least one of site information related to an examination purpose of the subject and region information in the medical image;
set a first region of interest corresponding to at least one of the site information related to the examination purpose of the subject and the region information in the medical image;
specify a second region of interest other than the first region of interest based on the first region of interest; and
generate a report regarding the second region of interest by using a generative model.

3. The medical image processing apparatus according to claim 2, wherein the processing circuitry is further configured to generate a report regarding the first region of interest by using the generative model.

4. The medical image processing apparatus according to claim 3, wherein the processing circuitry is further configured to generate a new report based on the report regarding the first region of interest and the report regarding the second region of interest.

5. The medical image processing apparatus according to claim 2, wherein
the processing circuitry is further configured to:
acquire imaging target information related to an imaging target of the medical image, and
specify the second region of interest based on an imaging target not included in the first region of interest in the imaging target information.

6. The medical image processing apparatus according to claim 5, wherein the processing circuitry is further configured to specify the second region of interest based on subject information related to the subject in addition to the imaging target information and the first region of interest.

7. The medical image processing apparatus according to claim 2, wherein the processing circuitry is further configured to set, based on information related to a symptom of the subject included in the site information, the first region of interest related to the symptom by using ontology.

8. The medical image processing apparatus according to claim 2, wherein the processing circuitry is further configured to:
perform image processing on the medical image,
set the first region of interest as a processing region in the medical image based on the region information, and
specify the second region of interest by causing the image processor to perform predetermined processing on the processing region.

9. The medical image processing apparatus according to claim 8, wherein the processing circuitry is further configured to receive an input related to the region information from a user.

10. The medical image processing apparatus according to claim 8, wherein the processing circuitry is further configured to acquire the region information based on the medical image and the site information by using a learning model configured to detect a site included in the medical image.

11. The medical image processing apparatus according to claim 2, wherein the processing circuitry is further configured to generate the report based on at least one of examination information related to an examination result of the subject and a user input in addition to the second region of interest.

12. The medical image processing apparatus according to claim 2, wherein the processing circuitry is further configured to:
cause an output device to output the report regarding the second region of interest, and
emphasize a portion related to the second region of interest in the report.

13. The medical image processing apparatus according to claim 2, wherein the processing circuitry is further configured to:
verify an interpreted image interpretation report that has been already interpreted based on the report regarding the second region of interest generated by a generator.

14. The medical image processing apparatus according to claim 13, wherein the processing circuitry is further configured to:
cause an output device to output a verification result of the interpreted image interpretation report obtained by a verifier, and
emphasize a difference between the verification result and the report generated by a generator.

15. The medical image processing apparatus according to claim 2, wherein the generative model is a multimodal model.

16. A medical image processing method for processing a medical image of a subject, the method comprising:
acquiring a medical image of the subject, and at least one of site information related to an examination purpose of the subject and region information in the medical image;
setting a first region of interest corresponding to at least one of the site information related to the examination purpose of the subject and the region information in the medical image;
specifying a second region of interest other than the first region of interest based on the first region of interest; and
generating a report regarding the second region of interest by using a generative model.
